(19) United States
(12) Patent Application Publication
(10) Pub. No.: US 2019/0270963 A1
(43) Pub. Date: Sep. 5, 2019

(54) METHOD TO SUPPRESS STEM CELL DIFFERENTIATION, METHOD TO PREPARE STEM CELLS, AND METHOD TO INDUCE DIFFERENTIATION OF STEM CELLS

(71) Applicant: UBE INDUSTRIES, LTD., Yamaguchi (JP)

(72) Inventors: Masahiko HAGIHARA, Yamaguchi (JP); Tetsuo KAWAGUCHI, Yamaguchi (JP); Kousuke BABA, Yamaguchi (JP)

(21) Appl. No.: 16/319,995

(22) PCT Filed: Jul. 25, 2017
(86) PCT No.: PCT/JP2017/026937

§ 371 Date: Jan. 23, 2019

(30) Foreign Application Priority Data
Jul. 25, 2016 (JP) 2016-145831

(51) Int. Cl. C12N 5/00; C12N 5/0735; C12N 5/074; C12N 5/0775; C12M 1/12

(52) U.S. Cl. CPC C12N 5/0068; C12N 5/0062; C12N 5/0606; C12M 25/14; C12N 5/0663; C12N 5/0018; C12N 5/0696

(57) ABSTRACT

The present invention relates to a method to suppress stem cell differentiation, said method including: (1) a step in which the stem cells are applied to a polymer porous film; and (2) a step in which the stem cells are cultivated and allowed to multiply, wherein the polymer porous film is a polymer porous film with a three-layer structure, having a surface layer A and a surface layer B that have a plurality of holes, and a macrovoid layer that is sandwiched between the surface layer A and the surface layer B, the average hole diameter of the holes present in the surface layer A is smaller than the average hole diameter of the holes present in the surface layer B, the macrovoid layer has dividing walls that are connected to the surface layers A and B, and a plurality of macrovoids that are surrounded by the dividing walls and the surface layers A and B, and the holes in the surface layers A and B are in communication with the macrovoids.

### FIELD

The present invention relates to a method for suppressing the differentiation of stem cells, a method for preparing stem cells, and a method for inducing the differentiation of stem cells.

### BACKGROUND

### Regarding Suppressing Differentiation of Stem Cells

As the importance of regenerative medicine has been acceleratingly increasing, the role played by stem cells is becoming increasingly important. For example, a large amount of stem cells is required when carrying out autologous cell transplantation. It is, however, known that stem cells tend to spontaneously differentiate during proliferation and it is difficult to culture stem cells while maintaining the undifferentiated state of the cells.

A method for culturing stem cells while suppressing differentiation using a cell culture substrate on which a protein belonging to the cadherin family or a fusion protein containing all or a part of a region of a protein belonging to the cadherin family and a polymer having a sugar side chain are immobilized or coated, has been reported (PTL 1).

In addition, methods of culturing stem cells while suppressing differentiation using hollow fibers have been reported (PTLs 2 and 3).

### Porous Polyimide Film

Porous polyimide films have been utilized in the prior art for filters and low permittivity films, and especially for battery-related purposes, such as fuel cell electrolyte membranes and the like. PTLs 4 to 6 describe porous polyimide films with numerous macro-voids, having excellent permeability for gases and the like, high porosity, excellent smoothness on both surfaces, relatively high strength and, despite high porosity, also excellent resistance against compression stress in the film thickness direction. All of these are porous polyimide films formed via amic acid.

A cell culturing method comprising steps of applying cells to a porous polyimide film and culturing them has been reported (PTL 7).

### PRIOR ART DOCUMENTS

### Patent Literature

PTL 1: JP2013-126405A

PTL 2: JP2014-60991A

PTL 3: JP2016-7207A

PTL 4: WO2010/038873

PTL 5: JP2011-219585A

PTL 6: JP2011-219586A

PTL 7: WO2015/012415

### DISCLOSURE OF THE INVENTION

### Problems to be Solved by the Invention

An object of the present invention is to provide a method capable of suppressing differentiation of stem cells using means totally different from conventional means so as to supply the stem cells in large amounts.

### Means for Solving the Problems

As a result of intensive studies in view of the above-mentioned object, the present inventors found that it is surprisingly possible to suppress differentiation of stem cells by culturing the stem cells on a three-layered porous polymer film having two surface layers with multiple pores and a macrovoid layer sandwiched between the two surface layers. This has led to the present invention.

Namely, the present invention includes the following aspects.
[1]

A method for suppressing the differentiation of stem cells, the method comprising the steps of:

(1) applying the stem cells to a porous polymer film; and

(2) culturing and proliferating the stem cells;

wherein the porous polymer film is a three-layer structure porous polymer film having a surface layer A and a surface layer B, the surface layers having a plurality of pores, and a macrovoid layer sandwiched between the surface layers A and B;

wherein an average pore diameter of the pores present in the surface layer A is smaller than an average pore diameter of the pores present in the surface layer B;

wherein the macrovoid layer has a partition wall bonded to the surface layers A and B, and a plurality of macrovoids surrounded by the partition wall and the surface layers A and B; and

wherein the pores in the surface layers A and B communicate with the macrovoids.
[2]

The method according to [1], wherein the cells are ES cells, EC cells, EG cells, nuclear transfer ES cells, ntES cells, iPS cells, hematopoietic stem cells, mesenchymal stem cells, hepatic stem cells, pancreatic stem cells, skin stem cells, bone marrow stem cells, muscle stem cells, germ stem cells, spermatogonia, type II alveolar epithelial cells, adipose stem cells, dental pulp stem cells, dedifferentiated adipocytes or MUSE cells.
[3]

The method according to [1] or [2], wherein the step (2) is carried out for at least 30 days.
[4]

The method according to any one of [1] to [3], wherein in the step (2), the cells are allowed to proliferate to 1.0×10⁵ or higher per square centimeter of the porous polymer film.
[5]

The method according to any one of [1] to [4], wherein two or more porous polymer films are layered either above and below or left and right in the cell culture medium.
[6]

The method according to any one of [1] to [5], wherein the porous polyimide film is
i) folded,
ii) wound into a roll-like shape,
iii) connected as sheets or pieces with a filamentous structure, or
iv) bound into a rope-like shape, and suspended or fixed in a cell culture medium in a cell culture vessel.
[7]

The method according to any one of [1] to [6], wherein, in step (2), a part or all of a porous polyimide film is not in contact with a liquid phase of a cell culture medium.
[8]

The method according to any one of [1] to [7], wherein in step (2), the total volume of the cell culture medium contained in the cell culture vessel is 10000 times or lower than the sum of the porous polyimide film volume comprising a cell viable region.
[9]

The method according to any one of [1] to [8], wherein an average pore diameter of the surface layer A is 0.01 to 50 μm.
[10]

The method according to any one of [1] to [9], wherein an average pore diameter of the surface layer B is 20 to 100 μm.
[11]

The method according to any one of [1] to [10], wherein a film thickness of the porous polymer film is 5 to 500 μm.
[12]

The method according to any one of [1] to [11], wherein the porous polymer film is a porous polyimide film.
[13]

The method according to [12], wherein the porous polyimide film is a porous polyimide film comprising a polyimide derived from tetracarboxylic dianhydride and diamine.
[14]

The method according to [12] or [13], wherein the porous polyimide film is a colored porous polyimide film that is obtained by molding a polyamic acid solution composition comprising a polyamic acid solution derived from tetracarboxylic dianhydride and diamine, and a coloring precursor, and subsequently heat-treating the resultant composition at 250° C. or higher.
[15]

The method according to any one of [1] to [11], wherein the porous polymer film is a porous polyethersulfone film.
[16]

A method for preparing stem cells, the method comprising the steps of:
(1) applying the stem cells to a porous polymer film; and
(2) culturing and proliferating the stem cells;

wherein the porous polymer film is a three-layer structure porous polymer film having a surface layer A and a surface layer B, the surface layers having a plurality of pores, and a macrovoid layer sandwiched between the surface layers A and B;

wherein an average pore diameter of the pores present in the surface layer A is smaller than an average pore diameter of the pores present in the surface layer B; wherein the macrovoid layer has a partition wall bonded to the surface layers A and B, and a plurality of macrovoids surrounded by the partition wall and the surface layers A and B; and

wherein the pores in the surface layers A and B communicate with the macrovoids; and

wherein in the step (2), the differentiation of the cells is suppressed.
[17]

A method for inducing the differentiation of stem cells, the method comprising the steps of:
(1) applying the stem cells to a porous polymer film;
(2) culturing and proliferating the stem cells; and
(3) culturing the cultured stem cells under differentiation-inducing conditions;

wherein the porous polymer film is a three-layer structure porous polymer film having a surface layer A and a surface layer B, the surface layers having a plurality of pores, and a macrovoid layer sandwiched between the surface layers A and B;

wherein an average pore diameter of the pores present in the surface layer A is smaller than an average pore diameter of the pores present in the surface layer B;

the macrovoid layer has a partition wall bonded to the surface layers A and B, and a plurality of macrovoids surrounded by the partition wall and the surface layers A and B;

wherein the pores in the surface layers A and B communicate with the macrovoids; and

wherein in the step (2), the differentiation of the stem cells is suppressed.

### Effects of the Invention

According to the present invention, it is possible to suppress differentiation of stem cells so as to supply the stem cells in large amounts.

### BRIEF DESCRIPTION OF DRAWINGS

FIG. 1 illustrates a model of cell culture using a porous polyimide film.

FIG. 2 illustrates an example of a cell culture system.

FIG. 3 depicts the results of gene analysis following culture of human mesenchymal stem cells on a porous polyimide film. In FIG. 3, the solid line represents results for cell samples treated by member culture for 3 days, 70 days, and 182 days. The broken line represents results for cell samples treated by normal culture for 3 days, 70 days, and 118 days.

FIG. 4 depicts a fluorescence microscopic image of staining with Oil red O of human mesenchymal stem cells which were cultured on a porous polyimide film for 6 days and then induced to differentiate into adipocytes for 13 days.

FIG. 5 depicts a fluorescence microscopic image of staining with BODIPY of human mesenchymal stem cells which were cultured on a porous polyimide film for 182 days and then induced to differentiate into adipocytes for 15 days.

FIG. 6 depicts time-dependent changes in the cell count when human mesenchymal stem cells were cultured with or without the use of a porous polyimide film.

FIG. 7 depicts time-dependent changes in the cell count during culture of human mesenchymal stem cells on a porous polyimide film.

FIG. 8 is a fluorescence microscopic image of staining with BODIPY of human mesenchymal stem cells which were cultured on a porous polyimide film for 125 days and then induced to differentiate for 10 days.

FIG. 9 shows time-dependent changes in the cell count during culture of human mesenchymal stem cells on a porous polyimide film.

FIG. 10 depicts fluorescence microscopic images of human mesenchymal stem cells on days 15, 30, and 150 after the start of culture on a porous polyimide film.

FIG. 11 depicts electron microscopic images of samples prepared by formalin-fixing a porous polyimide film on which human mesenchymal stem cells were engrafted.

FIG. 12 depicts fluorescence microscopic images of samples prepared by formalin-fixing a porous polyimide film on which human mesenchymal stem cells were engrafted.

FIG. 13 depicts time-dependent changes in the cell count during culture of human mesenchymal stem cells on a porous polyimide film for a long period of time.

FIG. 14 depicts a fluorescence microscopic image of staining with BODIPY of human mesenchymal stem cells which were cultured on a porous polyimide film for 463 days and then induced to differentiate into adipocytes for 26 days.

FIG. 15 depicts an optical microscopic image of a sample prepared by culturing human mesenchymal stem cells on a porous polyimide film for 490 days and then induced to differentiate into osteoblasts for 26 days, followed by mineralization induction.

FIG. 16A depicts the results of gene analysis following culture of human mesenchymal stem cells on a porous polyimide film for a long period of time.

FIG. 16B depicts the results of gene analysis following culture of human mesenchymal stem cells on a porous polyimide film for a long period of time.

FIG. 17 depicts time-dependent changes in the cell count during culture of human type II alveolar epithelial cells on a porous polyimide film.

FIG. 18 depicts the results of gene analysis following culture of human type II alveolar epithelial cells on a porous polyimide film.

FIG. 19 depicts an image of immunostaining of human type II alveolar epithelial cells on day 22 of culture. DAPI: blue, Pro SP-C: green, Podoplanin: red.

FIG. 20 depicts time-dependent changes in the cell count during culture of human type II alveolar epithelial cells on a porous polyimide film for a long period of time.

FIG. 21 depicts time-dependent changes in the cell counts during culture of human type II alveolar epithelial cells seeded on a porous polyimide film by different cell seeding methods.

FIG. 22 depicts the results of gene analysis following culture of human type II alveolar epithelial cells on a porous polyimide film for a long period of time.

### DESCRIPTION OF EMBODIMENTS

One aspect of the present invention relates to a method for suppressing the differentiation of stem cells, the method comprising the steps of:

(1) applying the stem cells to a porous polymer film; and

(2) culturing and proliferating the stem cells;

wherein the porous polymer film is a three-layer structure porous polymer film having a surface layer A and a surface layer B, the surface layers having a plurality of pores, and a macrovoid layer sandwiched between the surface layers A and B;

wherein an average pore diameter of the pores present in the surface layer A is smaller than an average pore diameter of the pores present in the surface layer B;

wherein the macrovoid layer has a partition wall bonded to the surface layers A and B, and a plurality of macrovoids surrounded by the partition wall and the surface layers A and B; and

wherein the pores in the surface layers A and B communicate with the macrovoids. This method is also referred to as the “method for suppressing the differentiation of the present invention” hereinbelow.

Another aspect of the present invention relates to a method for preparing stem cells, the method comprising the steps of:

(1) applying the stem cells to a porous polymer film; and

(2) culturing and proliferating the stem cells;

wherein the porous polymer film is a three-layer structure porous polymer film having a surface layer A and a surface layer B, the surface layers having a plurality of pores, and a macrovoid layer sandwiched between the surface layers A and B;

wherein an average pore diameter of the pores present in the surface layer A is smaller than an average pore diameter of the pores present in the surface layer B;

wherein the macrovoid layer has a partition wall bonded to the surface layers A and B, and a plurality of macrovoids surrounded by the partition wall and the surface layers A and B; and

wherein the pores in the surface layers A and B communicate with the macrovoids; and

wherein in the step (2), the differentiation of the cells is suppressed. This method is also referred to as the “method for preparing stem cells of the present invention” hereinbelow.

Another aspect of the present invention relates to a method for inducing the differentiation of stem cells, the method comprising the steps of:

(1) applying the stem cells to a porous polymer film;

(2) culturing and proliferating the stem cells; and

(3) culturing the cultured stem cells under differentiation-inducing conditions;

wherein the porous polymer film is a three-layer structure porous polymer film having a surface layer A and a surface layer B, the surface layers having a plurality of pores, and a macrovoid layer sandwiched between the surface layers A and B;

wherein an average pore diameter of the pores present in the surface layer A is smaller than an average pore diameter of the pores present in the surface layer B;

the macrovoid layer has a partition wall bonded to the surface layers A and B, and a plurality of macrovoids surrounded by the partition wall and the surface layers A and B;

wherein the pores in the surface layers A and B communicate with the macrovoids; and

wherein in the step (2), the differentiation of the stem cells is suppressed. This method is also referred to as the “differentiation induction method of the present invention” hereinbelow.

The “method for suppressing the differentiation of the present invention”, the “method for preparing stem cells of the present invention” and the “method for inducing the differentiation of the present invention” are also referred to as the “method of the present invention” hereinbelow.
1. Regarding Stem Cells Used in the Method of the Present Invention

The term “stem cell(s)” used herein refers to cell(s) having ability to divide to produce the same type of cells as themselves (self-renewal ability, i.e. self-replication ability) and ability to differentiate into a different type of cells. In recent years, it is possible to directly induce terminally differentiated cells into another terminally differentiated cells by a method such as direct reprogramming However, the “stem cells” described herein do not include terminally differentiated cells. Stem cells can be classified based on the following differences in differentiation ability.

### (1) Pluripotency

The term “pluripotency” used herein refers to ability to differentiate into all of cell lines belonging to the three germ layers (endoderm, mesoderm, and ectoderm). Examples thereof include, but are not limited to, embryonic stem cells (ES cells), embryonic carcinoma cells (EC cells), embryonic germ stem cells (EG cells), nuclear transfer ES cells, somatic cell-derived ES cells (ntES cells), induced pluripotent stem cells (iPS cells), and multi-lineage differentiating stress enduring (MUSE) cells.

### (2) Multipotency

The term “multipotency” used herein refers to ability to differentiate into various cell species, although differentiable cell lines are limited. In general, it is impossible to induce differentiation into other germ layers. However, there are exceptions. Multipotency means differentiation ability possessed by tissue stem cells (cells that are not terminally differentiated, which are present in the body of an organism). Examples of such cells include, but are not limited to, hematopoietic stem cells, mesenchymal stem cells, hepatic stem cells, pancreatic stem cells, skin stem cells, dental pulp stem cells, adipose stem cells, and dedifferentiated adipocytes.

### (3) Oligopotency

The term “oligopotency” used herein refers to ability to differentiate into several limited types of cell species. Cells having oligopotency are also referred to as progenitor cells, which are herein included in stem cells. Such cells are, but are not limited to, for example, bone marrow stem cells.

### (4) Unipotency

The term “unipotency” used herein refers to ability to differentiate into one limited type of differentiable cell species. Cells having unipotency are also referred to as progenitor cells, which are herein included in stem cells. Such cells can divide to proliferate as stem cells or differentiate and turn into cells of a different cell species (other than stem cells). Examples of such cells include, but are not limited to, for example, muscle stem cells, germ-line stem cells, spermatogonia cells, and type II alveolar epithelial cells.

Stem cells used in the method of the present invention are preferably, ES cells, EC cells, EG cells, nuclear transfer ES cells, ntES cells, iPS cells, hematopoietic stem cells, mesenchymal stem cells, hepatic stem cells, pancreatic stem cells, skin stem cells, bone marrow stem cells, muscle stem cells, germ-line stem cells, spermatogonia cells, or type II alveolar epithelial cells, and more preferably mesenchymal stem cells or type II alveolar epithelial cells.

Types of stem cells that can be used in the present invention are not particularly limited, but preferably mammalian stem cells, more preferably stem cells of primates (such as humans and monkeys), rodents (such as mice, rats, and guinea pigs), cats, dogs, rabbits, sheep, pigs, cattle, horses, donkeys, goats, or ferrets, and particularly preferably human stem cells.
2. Regarding a Step of Applying Stem Cells Used in the Present Invention to a Porous Polymer Film

A specific step of applying stem cells used in the present invention to a porous polymer film is not particularly limited. It is possible to adopt the step described herein or an arbitrary technique appropriate for applying stem cells to a film-like carrier. The method of the present invention encompasses, but is not limited to, for example, the following aspects to apply stem cells to a porous polymer film.

(A) An aspect comprising a step of seeding stem cells on a surface of the porous polymer film;

(B) An aspect comprising a step of placing a stem cell suspension liquid on a dry surface of the porous polymer film,

leaving the porous polymer film, moving the porous polymer film to facilitate the liquid to outflow, or stimulating a part of the surface to allow the film to absorb the stem cell suspension liquid, and

retaining stem cells in the stem cell suspension liquid inside of the film while allowing moisture to outflow; and

(C) An aspect comprising a step of moistening one side or both sides of the porous polymer film with a cell culture liquid or a sterilized liquid,

filling the moistened porous polymer film with a stem cell suspension liquid, and retaining stem cells in the stem cell suspension liquid inside of the film while allowing moisture to outflow.

The aspect (A) comprises directly seeding cell mass on a surface of a porous polymer film. Alternatively, an aspect in which a porous polymer film is placed in a stem cell suspension liquid, thereby impregnating the film with a cell culture liquid through a surface of the film is also included.

Stem cells seeded on the surface of the porous polymer film adhere to the porous polymer film so as to enter inside of porous cavities. Preferably, stem cells spontaneously adhere to the porous polymer film without externally applying physical or chemical force with a specific intention. It is possible to stably grow or proliferate stem cells seeded on the surface of the porous polymer film on the surface and/or inside of the film. Stem cells may be in various different forms depending on positions on a film for cell growth/proliferation.

In the aspect (B), a stem cell suspension liquid is placed on a dry surface of a porous polymer film. The stem cell suspension liquid is allowed to permeate inside of the film by leaving the porous polymer film, moving the porous polymer film to facilitate the liquid to outflow, or stimulating a part of the surface to allow the film to suction the stem cell suspension liquid. This is thought to be because of the nature derived from a surface form or the like of the porous polymer film, which is, however, not bound by theory. In this aspect, stem cells are suctioned at a site where the film is filled with the stem cell suspension liquid so as to be seeded.

Alternatively, as in the aspect (C), one side or both sides of the porous polymer film may be partially or entirely moistened with a cell culture liquid or sterilized liquid, and then, the moistened porous polymer film may be filled with a stem cell suspension liquid. In this case, the speed of passage of stem cell suspension liquid is remarkably improved.

For example, it is possible to employ a method for moistening only a part of a film in order to mainly prevent scattering of the film (hereinbelow described as “single-point wetting method”). The single-point wetting method is very close to a dry method in which a film is substantially not moistened (aspect (B)). It is, however, considered that the passage of cell fluid through a film becomes rapid at a small moistened portion. It is also possible to employ a method in which one side or both sides of a porous polymer film are entirely and sufficiently moistened (hereinbelow described as “wet film”) and the porous polymer film is filled with a stem cell suspension liquid (hereinbelow described as “wet film method”). In this case, the speed of passage of stem cell suspension liquid is remarkably improved across the porous polymer film.

In the aspects (B) and (C), stem cells in the stem cell suspension liquid are retained inside of the film while allowing moisture to outflow. This makes it possible to carry out a process of concentrating stem cells in the stem cell suspension liquid, a process of allowing unnecessary components other than stem cells to outflow with moisture, or a similar process.

The aspect (A) is sometimes referred to as “natural seeding” and the embodiments (B) and (C) are sometimes referred to as “suction seeding.”

Preferably, but not limited to, viable cells are selectively retained in a porous polymer film. Therefore, in preferred embodiments of the method of the present invention, viable cells are retained in the porous polymer film while dead cells preferentially outflow with moisture.

A sterilized liquid used in the aspect (C) is, but is not particularly limited to, a sterilized buffer solution or sterile water. The buffer solution is, for example, Dulbecco's PBS (+) or (−), a Hank's balanced salt solution (+) or (−), or the like. Examples of the buffer solution are listed in Table 1 below.

TABLE 1

| | Concentration | Concentration |
|---|---|---|
| Component | (mmol/L) | (g/L) |
| NaCl | 137 | 8.00 |
| KCl | 2.7 | 0.20 |
| Na₂HPO₄ | 10 | 1.44 |
| KH₂PO₄ | 1.76 | 0.24 |
| pH (—) | 7.4 | 7.4 |

Further, according to the method of the present invention, in order to apply stem cells to a porous polymer film, an aspect in which stem cells in a floating state are allowed to coexist with a porous polymer film by way of suspension so as to allow the stem cells to adhere to the film (tangling) is also included. For example, according to the method of the present invention, in order to apply stem cells to a porous polymer film, it is possible to put a cell culture medium, stem cells, and one or more porous polymer films described above into cell culture vessel. When the cell culture medium is in a liquid state, the porous polymer film is in a state of floating in the cell culture medium. The nature of the porous polymer film allows stem cells to adhere to the porous polymer film. Accordingly, it is possible to culture even stem cells, which are not originally suitable for floating culture, by allowing a porous polymer film to be in a state of floating in a cell culture medium. Preferably, stem cells spontaneously adhere to a porous polymer film. The expression “spontaneously adhere” means that stem cells are retained on a surface or inside of a porous polymer film without externally applying physical or chemical force with a specific intention.

To apply stem cells to a porous polymer film as described above, a combination of two or more types of methods may be used. For example, a combination of two or more methods in the aspects (A) to (C) may be used for applying stem cells to a porous polymer film.
3. Regarding a Step of Culturing and Proliferating Stem Cells Used in the Present Invention

Regarding cell culture, culture cells can be classified into adhesion culture cells and floating culture cells based on the existence form upon cell culture. Adhesion culture cells are culture cells which adhere to a culture vessel so as to proliferate, and medium exchange is carried out for subculture. Floating culture cells are culture cells which proliferate in a state of floating in a medium. Usually, dilution culture is performed for subculture without medium exchange. Since floating culture allows culture in a floating state, which means culture in a liquid, mass culture is possible. Therefore, floating culture is advantageous in that the number of cells that can be cultured per a unit space is larger than that during culture of adhering cells that grow only on a culture vessel surface because floating culture is three-dimensional culture.

According to the method of the present invention, when a porous polymer film is used in a state of floating in a cell culture medium, two or more pieces of the porous polymer film may be used. A porous polymer film is a three-dimensional flexible thin film. Therefore, for example, when pieces of the porous polymer film are used in a state of floating in a culture liquid, it becomes possible to bring a porous polymer film having a large surface area available for culture into a cell culture medium having a predetermined volume. In the case of normal culture, the vessel base area corresponds to the upper limit of an area available for cell culture. However, in the case of cell culture using the porous polymer film of the present invention, the entire large surface area of the porous polymer film brought into the medium can be an area available for cell culture. Since a porous polymer film allows a cell culture liquid to pass therethrough, it becomes possible to supply nutrients, oxygen, and the like into, for example, a folded film. In addition, totally unlike the conventional plane culture, a porous polymer film is a cell culture substrate having a three-dimensional flexible structure. Therefore, it is possible to culture adhesive cells in a culture vessel made of an arbitrary material having arbitrary shape and size regardless of the shape of the culture vessel (for example, a petri dish, flask, tank, bag, or the like).

Sizes and shapes of pieces of a porous polymer film are not particularly limited. The shapes may be arbitrary shapes such as circle, ellipse, square, triangle, polygon, and string-like shapes.

The porous polymer film of the present invention can be used in a deformed shape because of its flexibility. The porous polymer film may be formed into a three-dimensional shape but not a plane shape when used. For example, the porous polymer film may be i) folded, ii) wound into a roll-like shape, iii) connected as sheets or pieces with a filamentous structure, or iv) bound into a rope-like shape, and suspended or fixed in a cell culture medium in a cell culture vessel. By forming the shape as described in i) to iv), it is possible to put many porous polymer films in a cell culture medium with a certain volume as in the case of using pieces. Further, since individual pieces can be handled as an aggregate, cell bodies can be gathered to be transferred, resulting in highly comprehensive applicability.

Two or more porous polymer films may be layered either above and below or left and right in a cell culture medium as a concept similar to an aggregate of pieces. Layering porous polymer films also includes an aspect in which porous polymer films partially overlap. Multilayer culture makes it possible to culture stem cells in a small space at a high density. It is also possible to form a multilayer system including a different type of cells by further layering and placing films on a film on which stem cells have grown. The number of porous polymer films to be layered is not particularly limited.

According to the method of the present invention, preferably, stem cells grow and proliferate on a surface and inside of the porous polymer film.

According to the method of the present invention, stem cells can be cultured while suppressing differentiation for a long period of time which is at least 30 days, at least 60 days, at least 120 days, at least 200 days, or at least 300 days without conducting a subculture operation including trypsin treatment or the like as in the conventional case. In addition, according to the method of the present invention, stem cells can be cultured while suppressing differentiation for a period not shorter than a period during which culture can be carried out by conventional plane culture, for example, a period not less than 1.5 times, 2 times, 2.5 times, 3 times, 3.5 times, 4 times, or 4.5 times of the period of plane culture. According to the present invention, it is possible to maintain a life in an active state but not a dormant state without causing cell detachment or death or the like which occurs during long-term cell culture in a petri dish or the like for plane culture. In addition, according to the present invention, even stem cells after long-term culture remain substantially unchanged in terms of cell viability or the properties of stem cells (for example, the cell surface marker expression level and the like) as compared with stem cells before long-term culture. In addition, according to the present invention, stem cells three-dimensionally proliferate in a porous polymer film, which tends to prevent restriction of the culture area that may be seen conventional plane culture and contact inhibition that may occur due to a plane environment, thereby making it possible to conduct long-term culture for growth. Further, according to the present invention, by bringing additional porous polymer films into contact with a porous polymer film to which stem cells are adhering, it is possible to increase a space enabling cell culture as desired. Thus, it is possible to conduct long-term culture for growth while not conducting subculture involving trypsin treatment as in the conventional case and avoiding a confluent state which induces contact inhibition. Moreover, according to the present invention, a novel storage method in which stem cells are stored for a long period of time without freezing or the like is provided.
4. Regarding Porous Polymer Film Used in the Present Invention

An average pore diameter of the pore present on a surface layer A (hereinafter referred to as “surface A” or “mesh surface”) in the porous polymer film used for the present invention is not particularly limited, but is, for example, 0.01 μm or more and less than 200 μm, 0.01 to 150 μm, 0.01 to 100 μm, 0.01 to 50 μm, 0.01 to 40 μm, 0.01 to 30 μm, 0.01 to 20 μm, or 0.01 to 15 μm, preferably 0.01 to 15 μm.

The average pore diameter of the pore present on a surface layer B (hereinafter referred to as “surface B” or “large pore surface”) in the porous polymer film used for the present invention is not particularly limited so long as it is larger than the average pore diameter of the pore present on the surface A, but is, for example, greater than 5 μm and 200 μm or less, 20 μm to 100 μm, 30 μm to 100 μm, 40 μm to 100 μm, 50 μm to 100 μm, or 60 μm to 100 μm, preferably 20 μm to 100 μm.

The average pore diameter on the surface of the porous polymer film is determined by measuring pore area for 200 or more open pore portions, and calculated an average diameter according to the following Equation (1) from the average pore area assuming the pore shape as a perfect circle.

Average Pore Diameter=2×\sqrt{(s a/π)} (1)
(wherein Sa represents the average value for the pore areas)

The thicknesses of the surface layers A and B are not particularly limited, but is, for example, 0.01 to 50 μm, preferably 0.01 to 20 μm.

The average pore diameter of macrovoids in the planar direction of the film in the macrovoid layer in the porous polymer film is not particularly limited but is, for example, 10 to 500 μm, preferably 10 to 100 μm, and more preferably 10 to 80 μm. The thicknesses of the partition wall in the macrovoid layer are not particularly limited, but is, for example, 0.01 to 50 μm, preferably 0.01 to 20 μm. In an embodiment, at least one partition wall in the macrovoid layer has one or two or more pores connecting the neighboring macrovoids and having the average pore diameter of 0.01 to 100 μm, preferably 0.01 to 50 μm. In another embodiment, the partition wall in the macrovoid layer has no pore.

The film thickness of the porous polymer film used for the invention is not particularly limited, but may be 5 μm or more, 10 μm or more, 20 μm or more or 25 μm or more, and 500 μm or less, 300 μm or less, 100 μm or less, 75 μm or less, or 50 μm or less. It is preferably 5 to 500 μm, and more preferably 25 to 75 μm.

The film thickness of the porous polymer film used for the invention can be measured using a contact thickness gauge.

The porosity of the porous polymer film used in the present invention is not particularly limited but is, for example, 40% or more and less than 95%.

The porosity of the porous polymer film used for the invention can be determined by measuring the film thickness and mass of the porous film cut out to a prescribed size, and performing calculation from the basis weight according to the following Equation (2).

Porosity (%)=(1−2/(S×d×D)×100 (2)
(wherein S represents the area of the porous film, d represents the film thickness, w represents the measured mass, and D represents the polymer density. The density is defined as 1.34 g/cm³ when the polymer is a polyimide.)

The porous polymer film used for the present invention is preferably a porous polymer film which includes a three-layer structure porous polymer film having a surface layer A and a surface layer B, the surface layers having a plurality of pores, and a macrovoid layer sandwiched between the surface layers A and B; wherein the average pore diameter of the pore present on the surface layer A is 0.01 μm to 15 μm, and the average pore diameter of the pore present on the surface layer B is 20 μm to 100 μm; wherein the macrovoid layer has a partition wall bonded to the surface layers A and B, and a plurality of macrovoids surrounded by such a partition wall and the surface layers A and B, the thickness of the macrovoid layer, and the surface layers A and B is 0.01 to 20 μm; wherein the pores on the surface layers A and B communicate with the macrovoid, the total film thickness is 5 to 500 μm, and the porosity is 40% or more and less than 95%. In an embodiment, at least one partition wall in the macrovoide layer has one or two or more pores connecting the neighboring macrovoids with each other and having the average pore diameter of 0.01 to 100 μm, preferably 0.01 to 50 μm. In another embodiment, the partition wall does not have such pores.

The porous polymer film used for the present invention is preferably sterilized. The sterilization treatment is not particularly limited, but any sterilization treatment such as dry heat sterilization, steam sterilization, sterilization with a disinfectant such as ethanol, electromagnetic wave sterilization such as ultraviolet rays or gamma rays, and the like can be mentioned.

The porous polymer film used for the present invention is not particularly limited so long as it has the structural features described above and includes, preferably a porous polyimide film or porous polyethersulfone film (porous PES film).

Polyimide is a general term for polymers containing imide bonds in the repeating unit, and usually it refers to an aromatic polyimide in which aromatic compounds are directly linked by imide bonds. An aromatic polyimide has an aromatic-aromatic conjugated structure via an imide bond, and therefore has a strong rigid molecular structure, and since the imide bonds provide powerful intermolecular force, it has very high levels of thermal, mechanical and chemical properties.

The porous polyimide film usable for the present invention is a porous polyimide film preferably containing polyimide (as a main component) obtained from tetracarboxylic dianhydride and diamine, more preferably a porous polyimide film composed of tetracarboxylic dianhydride and diamine. The phrase “including as the main component” means that it essentially contains no components other than the polyimide obtained from a tetracarboxylic dianhydride and a diamine, as constituent components of the porous polyimide film, or that it may contain them but they are additional components that do not affect the properties of the polyimide obtained from the tetracarboxylic dianhydride and diamine.

In an embodiment, the porous polyimide film usable for the present invention includes a colored porous polyimide film obtained by forming a polyamic acid solution composition including a polyamic acid solution obtained from a tetracarboxylic acid component and a diamine component, and a coloring precursor, and then heat treating it at 250° C. or higher.

A polyamic acid is obtained by polymerization of a tetracarboxylic acid component and a diamine component. A polyamic acid is a polyimide precursor that can be cyclized to a polyimide by thermal imidization or chemical imidization.

The polyamic acid used may be any one that does not have an effect on the invention, even if a portion of the amic acid is imidized. Specifically, the polyamic acid may be partially thermally imidized or chemically imidized.

When the polyamic acid is to be thermally imidized, there may be added to the polyamic acid solution, if necessary, an imidization catalyst, an organic phosphorus-containing compound, or fine particles such as inorganic fine particles or organic fine particles. In addition, when the polyamic acid is to be chemically imidized, there may be added to the polyamic acid solution, if necessary, a chemical imidization agent, a dehydrating agent, or fine particles such as inorganic fine particles or organic fine particles. Even if such components are added to the polyamic acid solution, they are preferably added under conditions that do not cause precipitation of the coloring precursor.

In this specification, a “coloring precursor” is a precursor that generates a colored substance by partial or total carbonization under heat treatment at 250° C. or higher.

Coloring precursors usable for the production of the porous polyimide film are preferably uniformly dissolved or dispersed in a polyamic acid solution or polyimide solution and subjected to thermal decomposition by heat treatment at 250° C. or higher, preferably 260° C. or higher, even more preferably 280° C. or higher and more preferably 300° C. or higher, and preferably heat treatment in the presence of oxygen such as air, at 250° C., preferably 260° C. or higher, even more preferably 280° C. or higher and more preferably 300° C. or higher, for carbonization to produce a colored substance, more preferably producing a black colored substance, with carbon-based coloring precursors being most preferred.

The coloring precursor, when being heated, first appears as a carbonized compound, but compositionally it contains other elements in addition to carbon, and also includes layered structures, aromatic crosslinked structures and tetrahedron carbon-containing disordered structures.

Carbon-based coloring precursors are not particularly restricted, and for example, they include tar or pitch such as petroleum tar, petroleum pitch, coal tar and coal pitch, coke, polymers obtained from acrylonitrile-containing monomers, ferrocene compounds (ferrocene and ferrocene derivatives), and the like. Of these, polymers obtained from acrylonitrile-containing monomers and/or ferrocene compounds are preferred, with polyacrylonitrile being preferred as a polymer obtained from an acrylonitrile-containing monomer.

Moreover, in another embodiment, examples of the porous polyimide film which may be used for the preset invention also include a porous polyimide film which can be obtained by molding a polyamic acid solution derived from a tetracarboxylic acid component and a diamine component followed by heat treatment without using the coloring precursor.

The porous polyimide film produced without using the coloring precursor may be produced, for example, by casting a polyamic acid solution into a film, the polyamic acid solution being composed of 3 to 60% by mass of polyamic acid having an intrinsic viscosity number of 1.0 to 3.0 and 40 to 97% by mass of an organic polar solvent, immersing or contacting in a coagulating solvent containing water as an essential component, and imidating the porous film of the polyamic acid by heat treatment. In this method, the coagulating solvent containing water as an essential component may be water, or a mixed solution containing 5% by mass or more and less than 100% by mass of water and more than 0% by mass and 95% by mass or less of an organic polar solvent. Further, after the imidation, one surface of the resulting porous polyimide film may be subjected to plasma treatment.

The tetracarboxylic dianhydride which may be used for the production of the porous polyimide film may be any tetracarboxylic dianhydride, selected as appropriate according to the properties desired. Specific examples of tetracarboxylic dianhydrides include biphenyltetracarboxylic dianhydrides such as pyromellitic dianhydride, 3,3′,4,4′-biphenyltetracarboxylic dianhydride (s-BPDA) and 2,3,3′,4′-biphenyltetracarboxylic dianhydride (a-BPDA), oxydiphthalic dianhydride, diphenylsulfone-3,4,3′,4′-tetracarboxylic dianhydride, bis(3,4-dicarboxyphenyl)sulfide dianhydride, 2,2-bis(3,4-dicarboxyphenyl)-1,1,1,3,3,3-hexafluoropropane dianhydride, 2,3,3′,4′-benzophenonetetracarboxylic dianhydride, 3,3′,4,4′-benzophenonetetracarboxylic dianhydride, bis(3,4-dicarboxyphenyl)methane dianhydride, 2,2-bis(3,4-dicarboxyphenyl)propane dianhydride, p-phenylenebis(trimellitic acid monoester acid anhydride), p-biphenylenebis(trimellitic acid monoester acid anhydride), m-terphenyl-3,4,3′,4′-tetracarboxylic dianhydride, p-terphenyl-3,4,3′,4′-tetracarboxylic dianhydride, 1,3-bis(3,4-dicarboxyphenoxy)benzene dianhydride, 1,4-bis(3,4-dicarboxyphenoxy)benzene dianhydride, 1,4-bis(3,4-dicarboxyphenoxy)biphenyl dianhydride, 2,2-bis[(3,4-dicarboxyphenoxy)phenyl]propane dianhydride, 2,3,6,7-naphthalenetetracarboxylic dianhydride, 1,4,5,8-naphthalenetetracarboxylic dianhydride, 4,4′-(2,2-hexafluoroisopropylidene)diphthalic dianhydride, and the like. Also preferably used is an aromatic tetracarboxylic acid such as 2,3,3′,4′-diphenylsulfonetetracarboxylic acid. These may be used alone or in appropriate combinations of two or more.

Particularly preferred among these are at least one type of aromatic tetracarboxylic dianhydride selected from the group consisting of biphenyltetracarboxylic dianhydride and pyromellitic dianhydride. As a biphenyltetracarboxylic dianhydride there may be suitably used 3,3′,4,4′-biphenyltetracarboxylic dianhydride.

As diamine which may be used for the production of the porous polyimide film, any diamine may be used. Specific examples of diamines include the following.

1) Benzenediamines with one benzene nucleus, such as 1,4-diaminobenzene(paraphenylenediamine), 1,3-diaminobenzene, 2,4-diaminotoluene and 2,6-diaminotoluene;

2) diamines with two benzene nuclei, including diaminodiphenyl ethers such as 4,4′-diaminodiphenyl ether and 3,4′-diaminodiphenyl ether, and 4,4′-diaminodiphenylmethane, 3,3′-dimethyl-4,4′-diaminobiphenyl, 2,′-dimethyl-4,4′-diaminobiphenyl, 2,2′-bis(trifluoromethyl)-4,4′-diaminobiphenyl, 3,3′-dimethyl-4,4′-diaminodiphenylmethane, 3,3′-dicarboxy-4,4′-diaminodiphenylmethane, 3,3′,5,5′-tetramethyl-4,4′-diaminodiphenylmethane, bis(4-aminophenyl)sulfide, 4,4′-diaminobenzanilide, 3,3′-dichlorobenzidine, 3,3′-dimethylbenzidine, 2,2′-dimethylbenzidine, 3,3′-dimethoxybenzidine, 2,2′-dimethoxybenzidine, 3,3′-diaminodiphenyl ether, 3,4′-diaminodiphenyl ether, 4,4′-diaminodiphenyl ether, 3,3′-diaminodiphenyl sulfide, 3,4′-diaminodiphenyl sulfide, 4,4′-diaminodiphenyl sulfide, 3,3′-diaminodiphenylsulfone, 3,4′-diaminodiphenylsulfone, 4,4′-diaminodiphenylsulfone, 3,3′-diaminobenzophenone, 3,3′-diamino-4,4′-dichlorobenzophenone, 3,3′-diamino-4,4′-dimethoxybenzophenone, 3,3′-diaminodiphenylmethane, 3,4′-diaminodiphenylmethane, 4,4′-diaminodiphenylmethane, 2,2-bis(3-aminophenyl)propane, 2,2-bis(4-aminophenyl)propane, 2,2-bis(3-aminophenyl)-1,1,1,3,3,3-hexafluoropropane, 2,2-bis(4-aminophenyl)-1,1,1,3,3,3-hexafluoropropane, 3,3′-diaminodiphenyl sulfoxide, 3,4′-diaminodiphenyl sulfoxide and 4,4′-diaminodiphenyl sulfoxide;

3) diamines with three benzene nuclei, including 1,3-bis(3-aminophenyl)benzene, 1,3-bis(4-aminophenyl)benzene, 1,4-bis(3-aminophenyl)benzene, 1,4-bis(4-aminophenyl)benzene, 1,3-bis(4-aminophenoxy)benzene, 1,4-bis(3-aminophenoxy)benzene, 1,4-bis(4-aminophenoxy)benzene, 1,3-bis(3-aminophenoxy)-4-trifluoromethylbenzene, 3,3′-diamino-4-(4-phenyl)phenoxybenzophenone, 3,3′-diamino-4,4′-di(4-phenylphenoxy)benzophenone, 1,3-bis(3-aminophenyl sulfide)benzene, 1,3-bis(4-aminophenyl sulfide)benzene, 1,4-bis(4-aminophenyl sulfide)benzene, 1,3-bis(3-aminophenylsulfone)benzene, 1,3-bis(4-aminophenylsulfone)benzene, 1,4-bis(4-aminophenylsulfone)benzene, 1,3-bis[2-(4-aminophenyl)isopropyl]benzene, 1,4-bis[2-(3-aminophenyl)isopropyl]benzene and 1,4-bis[2-(4-aminophenyl)isopropyl]benzene;

4) diamines with four benzene nuclei, including 3,3′-bis(3-aminophenoxy)biphenyl, 3,3′-bis(4-aminophenoxy)biphenyl, 4,4′-bis(3-aminophenoxy)biphenyl, 4,4′-bis(4-aminophenoxy)biphenyl, bis[3-(3-aminophenoxy)phenyl]ether, bis[3-(4-aminophenoxy)phenyl]ether, bis[4-(3-aminophenoxy)phenyl]ether, bis [4-(4-aminophenoxy)phenyl]ether, bis[3-(3-aminophenoxy)phenyl]ketone, bis[3-(4-aminophenoxy)phenyl]ketone, bis[4-(3-aminophenoxy)phenyl]ketone, bis[4-(4-aminophenoxy)phenyl]ketone, bis[3-(3-aminophenoxy)phenyl] sulfide, bis[3-(4-aminophenoxy)phenyl] sulfide, bis[4-(3-aminophenoxy)phenyl] sulfide, bis[4-(4-aminophenoxy)phenyl] sulfide, bis[3-(3-aminophenoxy)phenyl]sulfone, bis[3-(4-aminophenoxy)phenyl] sulfone, bis [4-(3-aminophenoxy)phenyl] sulfone, bis[4-(4-aminophenoxy)phenyl]sulfone, bis[3-(3-aminophenoxy)phenyl]methane, bis[3-(4-aminophenoxy)phenyl]methane, bis[4-(3-aminophenoxy)phenyl]methane, bis[4-(4-aminophenoxy)phenyl]methane, 2,2-bis[3-(3-aminophenoxy)phenyl]propane, 2,2-bis[3-(4-aminophenoxy)phenyl]propane, 2,2-bis[4-(3-aminophenoxy)phenyl]propane, 2,2-bis[4-(4-aminophenoxy)phenyl]propane, 2,2-bis[3-(3-aminophenoxy)phenyl]-1,1,1,3,3,3-hexafluoropropane, 2,2-bis[3-(4-aminophenoxy)phenyl]-1,1,1,3,3,3-hexafluoropropane, 2,2-bis[4-(3-aminophenoxy)phenyl]-1,1,1,3,3,3-hexafluoropropane and 2,2-bis[4-(4-aminophenoxy)phenyl]-1,1,1,3,3,3-hexafluoropropane.

These may be used alone or in mixtures of two or more. The diamine used may be appropriately selected according to the properties desired.

Preferred among these are aromatic diamine compounds, with 3,3′-diaminodiphenyl ether, 3,4′-diaminodiphenyl ether, 4,4′-diaminodiphenyl ether, paraphenylenediamine, 1,3-bis(3-aminophenyl)benzene, 1,3-bis(4-aminophenyl)benzene, 1,4-bis(3-aminophenyl)benzene, 1,4-bis(4-aminophenyl)benzene, 1,3-bis(4-aminophenoxy)benzene and 1,4-bis(3-aminophenoxy)benzene being preferred for use. Particularly preferred is at least one type of diamine selected from the group consisting of benzenediamines, diaminodiphenyl ethers and bis(aminophenoxy)phenyl.

From the viewpoint of heat resistance and dimensional stability under high temperature, the porous polyimide film which may be used for the invention is preferably formed from a polyimide obtained by combination of a tetracarboxylic dianhydride and a diamine, having a glass transition temperature of 240° C. or higher, or without a distinct transition point at 300° C. or higher.

From the viewpoint of heat resistance and dimensional stability under high temperature, the porous polyimide film which may be used for the invention is preferably a porous polyimide film comprising one of the following aromatic polyimides:

(i) An aromatic polyimide comprising at least one tetracarboxylic acid unit selected from the group consisting of biphenyltetracarboxylic acid units and pyromellitic acid units, and an aromatic diamine unit,

(ii) an aromatic polyimide comprising a tetracarboxylic acid unit and at least one type of aromatic diamine unit selected from the group consisting of benzenediamine units, diaminodiphenyl ether units and bis(aminophenoxy)phenyl units, and/or,

(iii) an aromatic polyimide comprising at least one type of tetracarboxylic acid unit selected from the group consisting of biphenyltetracarboxylic acid units and pyromellitic acid units, and at least one type of aromatic diamine unit selected from the group consisting of benzenediamine units, diaminodiphenyl ether units and bis(aminophenoxy)phenyl units.

The porous polyimide film used in the present invention is preferably a three-layer structure porous polyimide film having a surface layer A and a surface layer B, the surface layers having a plurality of pores, and a macrovoid layer sandwiched between the surface layers A and B; wherein an average pore diameter of the pores present in the surface layer A is 0.01 μm to 15 μm, and the mean pore diameter present on the surface layer B is 20 μm to 100 μm; wherein the macrovoid layer has a partition wall bonded to the surface layers A and B, and a plurality of macrovoids surrounded by such a partition wall and the surface layers A and B; wherein the thickness of the macrovoid layer, and the surface layers A and B is 0.01 to 20 μm, wherein the pores on the surface layers A and B communicate with the macrovoid, the total film thickness is 5 to 500 μm, and the porosity is 40% or more and less than 95%. In this case, at least one partition wall in the macrovoid layer has one or two or more pores connecting the neighboring macrovoids and having the average pore diameter of 0.01 to 100 μm, preferably 0.01 to 50 μm.

For example, porous polyimide films described in WO2010/038873, Japanese Unexamined Patent Publication (Kokai) No. 2011-219585 or Japanese Unexamined Patent Publication (Kokai) No. 2011-219586 may be used for the present invention.

The porous PES film which may be used for the present invention contains polyethersulfone and typically consists substantially of polyethersulfone. Polyethersulfone may be synthesized by the method known to those skilled in the art. For example, it may be produced by a method wherein a dihydric phenol, an alkaline metal compound and a dihalogenodiphenyl compound are subjected to polycondensation reaction in an organic polar solvent, a method wherein an alkaline metal di-salt of a dihydric phenol previously synthesized is subjected to polycondensation reaction dihalogenodiphenyl compound in an organic polar solvent or the like.

Examples of an alkaline metal compound include alkaline metal carbonate, alkaline metal hydroxide, alkaline metal hydride, alkaline metal alkoxide and the like. Particularly, sodium carbonate and potassium carbonate are preferred.

Examples of a dihydric phenol compound include hydroquinone, catechol, resorcin, 4,4′-biphenol, bis (hydroxyphenyl)alkanes (such as 2,2-bis(hydroxyphenyl)propane, and 2,2-bis(hydroxyphenyl)methane), dihydroxydiphenylsulfones, dihydroxydiphenyl ethers, or those mentioned above having at least one hydrogen on the benzene rings thereof substituted with a lower alkyl group such as a methyl group, an ethyl group, or a propyl group, or with a lower alkoxy group such as a methoxy group, or an ethoxy group. As the dihydric phenol compound, two or more of the aforementioned compounds may be mixed and used.

Polyethersulfone may be a commercially available product. Examples of a commercially available product include SUMIKAEXCEL 7600P, SUMIKAEXCEL 5900P (both manufactured by Sumitomo Chemical Company, Limited).

The logarithmic viscosity of the polyethersulfone is preferably 0.5 or more, more preferably 0.55 or more from the viewpoint of favorable formation of a macrovoid of the porous polyethersulfone film; and it is preferably 1.0 or less, more preferably 0.9 or less, further preferably 0.8 or less, particularly preferably 0.75 or less from the viewpoint of the easy production of a porous polyethersulfone film.

Further, from the viewpoints of heat resistance and dimensional stability under high temperature, it is preferred that the porous PES film or polyethersulfone as a raw material thereof has a glass transition temperature of 200° C. or higher, or that a distinct glass transition temperature is not observed.

The method for producing the porous PES film which may be used for the present invention is not particularly limited. For example, the film may be produced by a method including the following steps:

a step in which polyethersulfone solution containing 0.3 to 60% by mass of polyethersulfone having logarithmic viscosity of 0.5 to 1.0 and 40 to 99.7% by mass of an organic polar solvent is casted into a film, immersed in or contacted with a coagulating solvent containing a poor solvent or non-solvent of polyethersulfone to produce a coagulated film having pores; and

a step in which the coagulated film having pores obtained in the above-mentioned step is heat-treated for coarsening of the aforementioned pores to obtain a porous PES film;

wherein the heat treatment includes the temperature of the coagulated film having the pores is raised higher than the glass transition temperature of the polyethersulfone, or up to 240° C. or higher.

The porous PES film which can be used in the present invention is preferably a porous PES film having a surface layer A, a surface layer B, and a macrovoid layer sandwiched between the surface layers A and B,

wherein the macrovoid layer has a partition wall bonded to the surface layers A and B, and a plurality of macrovoids surrounded by such a partition wall and the surface layers A and B, the macrovoids having the average pore diameter in the planar direction of the film of 10 to 500 μm;

wherein the thickness of the macrovoid layer is 0.1 to 50 μm,

each of the surface layers A and B has a thickness of 0.1 to 50 μm,

wherein one of the surface layers A and B has a plurality of pores having the average pore diameter of more than 5 μm and 200 μm or less, while the other has a plurality of pores having the average pore diameter of 0.01 μm or more and less than 200 μm,

wherein one of the surface layers A and B has a surface aperture ratio of 15% or more while other has a surface aperture ratio of 10% or more,

wherein the pores of the surface layers A and B communicate with the macrovoids,

wherein the porous PES film has total film thickness of 5 to 500 μm and a porosity of 50 to 95%.
5. Cell Culture and Culture Volume

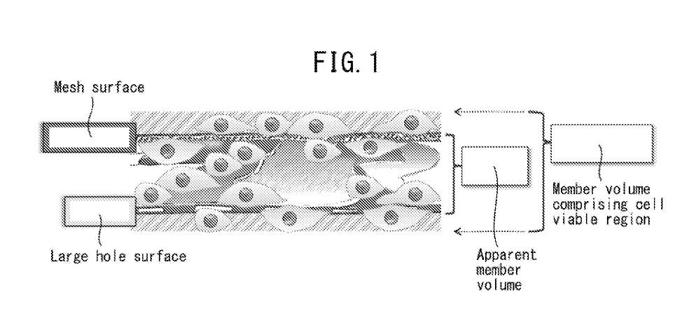

FIG. 1 illustrates a model of cell culture using a polymer porous film. FIG. 1 is a drawing for helping to understand, and each component is not illustrated in actual size. According to the method of the present invention, stem cells are applied to a porous polymer film so as to be cultured thereon such that stem cells grow in large amounts in multilaterally connected porous parts inside of the porous polymer film or on the surface thereof, thereby making it possible to conveniently culture stem cells in large amounts. In addition, according to the method of the present invention, it is possible to culture stem cells in large amounts while significantly reducing the amount of a medium used for cell culture as compared with that in the conventional method. For example, culture of stem cells in large amounts is possible for a long period of time even in a state in which a part or all of a porous polymer film is not in contact with a liquid phase of a cell culture medium. In addition, it is also possible to significantly reduce the total volume of the cell culture medium contained in the cell culture vessel relative to the sum of the porous polymer film volume comprising a cell viable region.

The volume occupied by the porous polymer film free of cells in the space including the volume of the internal gap defined herein is referred to as “apparent porous polymer film volume” (see FIG. 1). In addition, in a state in which stem cells are applied to a porous polymer film so that the stem cells are supported on the surface and inside of the porous polymer film, the volume occupied by the porous polymer film, the stem cells, and a medium infiltrating into the porous polymer film as a whole in the space is referred to as “porous polymer film volume comprising a cell viable region” (see FIG. 1). In the case of a porous polymer film having a film thickness of 25 μm, the porous polymer film volume comprising a cell viable region is a value greater by approximately 50% at maximum than the apparent porous polymer film volume. According to the method of the present invention, it is possible to accommodate a plurality of porous polymer films in a single cell culture vessel to conduct culture. In this case, the sum of the porous polymer film volume comprising a cell viable region for each of the plurality of porous polymer films supporting stem cells may be simply referred to as “sum of the porous polymer film volume comprising a cell viable region.”

By the method of the present invention, it is possible to culture stem cells in a favorable manner for a long period of time even under conditions in which the total volume of the cell culture medium contained in the cell culture vessel is 10000 times or less than the sum of the porous polymer film volume comprising a cell viable region. In addition, it is possible to culture stem cells in a favorable manner for a long period of time even under conditions in which the total volume of the cell culture medium contained in the cell culture vessel is 1000 times or less than the sum of the porous polymer film volume comprising a cell viable region. Further, it is possible to culture stem cells in a favorable manner for a long period of time even under conditions in which the total volume of the cell culture medium contained in the cell culture vessel is 100 times or less than the sum of the porous polymer film volume comprising a cell viable region. Furthermore, it is possible to culture stem cells in a favorable manner for a long period of time even under conditions in which the total volume of the cell culture medium contained in the cell culture vessel is 10 times or less than the sum of the porous polymer film volume comprising a cell viable region.

In other words, according to the present invention, it becomes possible to miniaturize the space (vessel) for performing cell culture to the limit as compared with conventional cell culture systems performing two-dimensional culture. In addition, when it is desired to increase the number of stem cells to be cultured, it becomes possible to increase the volume for cell culture in a flexible manner by a convenient operation such as increasing the number of porous polymer films to be layered. A cell culture system provided with porous polymer films used in the present invention makes it possible to separate a space (vessel) for culturing stem cells from a space (vessel) for storing a cell culture medium, thereby allowing preparation of a cell culture medium in a necessary amount depending on the number of cells to be cultured. The volume of a space (vessel) for storing a cell culture medium may be increased or decreased depending on the purpose. Alternatively, a replaceable vessel may be used without particular limitations.

According to the method of the present invention, for example, culture is performed to an extent that the number of stem cells contained in a cell culture vessel after culture using a porous polymer film is 1.0×10⁵ cells or more, 1.0×10⁶ cells or more, 2.0×10⁶ cells or more, 5.0×10⁶ cells or more, 1.0×10⁷ cells or more, 2.0×10⁷ cells or more, 5.0×10⁷ cells or more, 1.0×10⁸ cells or more, 2.0×10⁸ cells or more, 5.0×10⁸ cells or more, 1.0×10⁹ cells or more, 2.0×10⁹ cells or more, or 5.0×10⁹ cells or more per 1 milliliter of the medium, provided that all of the stem cells are uniformly dispersed in the cell culture medium contained in the cell culture vessel.

As a method for measuring the number of cells during or after culture, various known methods can be used. For example, as the method in which the number of stem cells contained in a cell culture vessel after culture using a porous polymer film is measured, provided that all of the stem cells are uniformly dispersed in a cell culture medium contained in the cell culture vessel, known methods can be used if appropriate. For example, a method for measuring the number of cells using CCK 8 can be used if appropriate. Specifically, the cell count in normal culture without the use of a porous polymer film is measured using Cell Counting Kit8 which is a solution reagent manufactured by DOJINDO LABORATORIES (hereinbelow described as “CCK8”) to calculate a correlation coefficient between absorbance and the actual cell count. Thereafter, a porous polymer film to which stem cells have been applied to be cultured is transferred to a medium containing CCK8 and stored in an incubator for 1 to 3 hours, the supernatant is extracted to measure absorbance at a wavelength of 480 nm, and the cell count is calculated based on the correlation coefficient obtained in advance.

From another viewpoint, mass culture of cells means, for example, culture during which the cell count per square centimeter of a porous polymer film reaches 1.0×10⁵ cells or more, 2.0×10⁵ cells or more, 1.0×10⁶ cells or more, 2.0×10⁶ cells or more, 5.0×10⁶ cells or more, 1.0×10⁷ cells or more, 2.0×10⁷ cells or more, 5.0×10⁷ cells or more, 1.0×10⁸ cells or more, 2.0×10⁸ cells or more, or 5.0×10⁸ cells or more after culture with the use of the porous polymer film. The cell count per square centimeter of the porous polymer film can be determined by known means such as a cell counter if appropriate.
6. Culture System and Culture Conditions for Stem Cells

According to the method of the present invention, a culture system and culture conditions for stem cells can be determined depending on types of stem cells if appropriate. There are known culture methods suitable for various stem cells. A person skilled in the art can culture stem cells which are applied to a porous polymer film by any known method. A cell culture medium can also be prepared depending on types of stem cells.

For example, a cell culture medium is listed on the cell culture medium catalog of LONZA. A cell culture medium that can be used in the method of the present invention may be in any form such as a liquid medium, a semi-solid medium, or a solid medium. In addition, a liquid medium in the droplet form is sprayed into a cell culture vessel, thereby allowing the medium to be in contact with a porous polymer film supporting cells.

Regarding cell culture using a porous polymer film, a porous polymer film can coexist with other floating type culture carriers such as a microcarrier and cellulose sponge.

According to the method of the present invention, the shape, scale, and the like of the system used for culture are not particularly limited, and a system ranging from a small system such as a petri dish, a flask, a plastic bag, or a test tube for cell culture to a large system such as large tank can be appropriately used. For example, a BD Falcon cell culture dish manufactured by Becton, Dickinson and Company, Nunc Cell Factory Systems manufactured by Thermo Fisher Scientific, Inc., and the like are included. By using a porous polymer film in the present invention, it becomes possible to perform culture in a state similar to floating culture using a system for floating culture even with cells that were originally unable to be cultured by floating culture. As a system for floating culture, for example, a spinner flask system manufactured by Corning Incorporated or a rotary culture system can be used. In addition, as an environment that can achieve similar functions, a hollow fiber culture system such as Fiber Cell (registered trademark) System of Veritas Technologies can also be used.

It is also possible to perform culture according to the method of the present invention in a manner such that a porous polymer film sheet is exposed in the air using a continuous circulation or an open type apparatus for continuously adding and recovering a culture medium on a porous polymer film.

According to the present invention, cell culture may be performed in a system in which a cell culture medium is continuously or intermittently supplied from cell culture medium supply means installed outside of a cell culture vessel to the cell culture vessel. At such time, a cell culture medium may be circulated between the cell culture medium supply means and the cell culture vessel in the system.

When cell culture is performed in a system in which a cell culture medium is continuously or intermittently supplied from cell culture medium supply means installed outside of a cell culture vessel to the cell culture vessel, the system may be a cell culture system comprising a culture unit as a cell culture vessel and a medium supply unit as cell culture medium supply means,

wherein the culture unit is a culture unit that accommodates one or more porous polymer films for supporting cells and is provided with a medium supply inlet and a medium discharge outlet; and

wherein the medium supply unit is a medium supply unit that is provided with a medium accommodating vessel, a medium supply line, and a liquid feeding pump that continuously or intermittently conducts liquid feeding of a medium via the medium supply line, in which a first end portion of the medium supply line is in contact with the medium in the medium accommodating vessel, and a second end portion of the medium supply line communicates via the medium supply inlet of the culture unit with the interior of the culture unit.

In addition, in the above-mentioned cell culture system, the culture unit may be a culture unit that is not provided with an air supply inlet, an air discharge outlet, and an oxygen exchange membrane, and it may also be a culture unit that is provided with an air supply inlet and an air discharge outlet or an oxygen exchange membrane. Even when the culture unit is not provided with an air supply inlet, an air discharge outlet, and an oxygen exchange membrane, oxygen and the like necessary for cell culture are sufficiently supplied to cells via the medium. Further, in the above-mentioned cell culture system, the culture unit may be further provided with a medium discharge line such that a first end portion of the medium discharge line is connected to the medium accommodating vessel, a second end portion of the medium discharge line communicates with the interior of the culture unit via the medium discharge outlet of the culture unit, and the medium can be circulated between the medium supply unit and the culture unit.

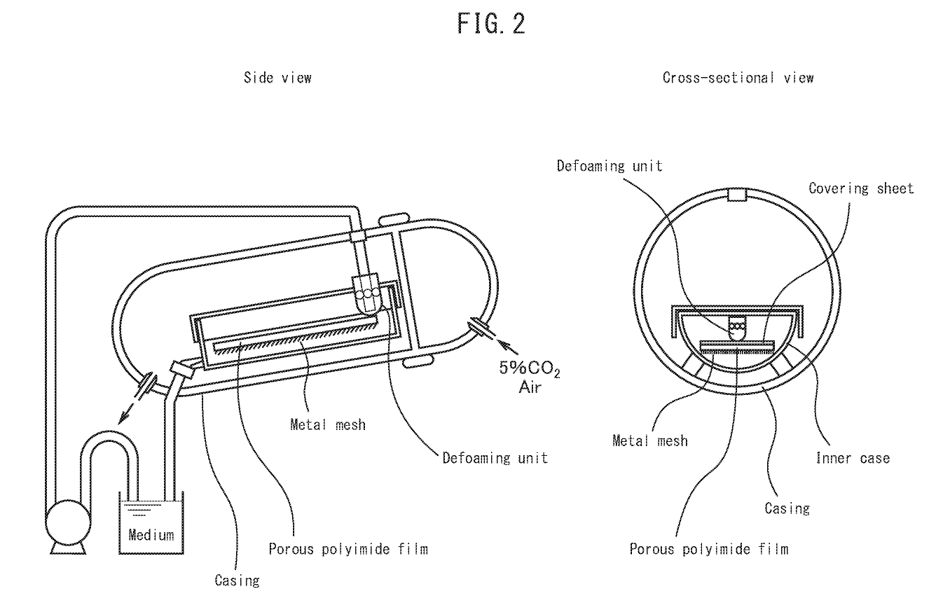

FIG. 2 illustrates an example of a cell culture system as an example of the above-mentioned cell culture system. However, a cell culture system that can be used for the object of the present invention is not limited thereto.
7. Regarding the Differentiation Induction Method of the Present Invention

According to the differentiation induction method of the present invention, stem cells that have proliferated on a porous polymer film can be induced to differentiate when cultured under differentiation inductions. Specific steps concerning differentiation induction of stem cells are not particularly limited. It is possible to adopt an arbitrary technique suitable for allowing stem cells that have grown on a film-like carrier to be in contact with a differentiation induction environment such as a differentiation induction medium, including the steps described herein.

It is possible to use, for example, factors such as a nerve growth factor (NGF) and retinoic acid for differentiation from pluripotent stem cells into tissue stem cells, although it is not limited. As the medium, an aMEM medium in the coexistence of 10% FBS or the like can be used.

It is possible to use factors such as basic fibroblast growth factors and forskolin or neuregulin for differentiation from tissue stem cells to cells of each tissue, although it is not limited. As the medium, a usual MEM medium or the like can be used, although it is not limited.

The present invention will now be explained in greater detail by Examples. It is to be understood, however, that the invention is not limited to these Examples. A person skilled in the art may easily implement modifications and changes to the invention based on the description in the present specification, and these are also encompassed within the technical scope of the invention.

### EXAMPLES

The porous polyimide films used in the following examples were prepared by forming a polyamic acid solution composition including a polyamic acid solution obtained from 3,3′,4,4′-biphenyltetracarboxylic dianhydride (s-BPDA) as a tetracarboxylic acid component and 4,4′-diaminodiphenyl ether (ODA) as a diamine component, and polyacrylamide as a coloring precursor, and performing heat treatment at 250° C. or higher. The resulting porous polyimide film was a three-layer structure porous polyimide film having a surface layer A and a surface layer B, the surface layers having a plurality of pores, and a macrovoid layer sandwiched between the surface layers A and B; wherein the average pore diameter of the pore present on the surface layer A was 6 μm, the average pore diameter of the pore present on the surface layer B was 46 μm, and the film thickness was 25 μm, and the porosity was 73%.

### Example 1

Gene Analysis of Human Mesenchymal Stem Cells Cultured on Porous Polyimide Film
1. Sample Preparation

To a sterilized 2 cm×2 cm square vessel (Thermo Fisher Scientific, Inc., cat. 103), 0.5 ml of a stem cell culture medium (manufactured by LONZA, PT-3001) was added, and a sterilized 1.4 cm size square porous polyimide film was immersed therein such that A-surface of the mesh structure of the film was faced up. Human mesenchymal stem cells were seeded at a density of 4×10⁴ cells per porous polyimide film, and the cells were continuously cultured in a CO₂ incubator while conducting medium exchange twice weekly. Cells cultured in the vessel for 3 days, 70 days, and 182 days were respectively used as gene analysis samples. The above-mentioned culture with the use of a porous polyimide film is hereinbelow referred to as “member culture,” and the obtained cell sample is hereinbelow referred to as “member culture cell sample.”

In addition, mesenchymal stem cells were cultured on a PLL-coated petri dish (manufactured by Sumitomo Bakelite Company Limited.; internal diameter: 10 cm) under the same conditions except that no porous polyimide film was used. The cells cultured for 3 days, 70 days, and 118 days were respectively used as gene analysis samples. The above-mentioned culture without the use of a porous polyimide film is hereinbelow referred to as “normal culture,” and the obtained cell sample is hereinbelow referred to as “normal culture cell sample.”
2. Gene Analysis

Gene analysis was conducted for the obtained samples by the following procedures.
(1) RNA Extraction

RNA was extracted using an RNeasy Plus micro Kit (Qiagen) according to the attached protocol. RNA was extracted with 30 μL of Nuclease Free Water, and then, decomposition treatment of genomic DNA with DNase was carried out using a TURBO DNA free Kit (Life Technologies). After decomposition treatment, the concentration of the RNA solution was measured by Nano Drop 2000 (Thermo Fishier Scientific).
(2) cDNA Synthesis

After measurement of the concentration, the RNA solution was adjusted to 12.5 ng/μL, and 100 ng thereof was used as a template to conduct cDNA synthesis. For synthesis, SuperScript (trademark) III First-Strand Synthesis System for RT-PCR (Life Technologies) was used. The concentration of the cDNA solution was measured by Nano Drop 2000.
(3) q-PCR Reaction

The cDNA solution was adjusted to 200 ng/μL, and 200 ng thereof was used as a template to conduct measurement by real-time PCR. PCR was conducted using CFX connect (Bio-Rad), and SsoAdvanced (trademark) Universal SYBR Green Supermix (Bio-Rad) was used as a reagent. The expression levels of mesenchymal stem cell-positive markers (CD166, CD44, CD105, CD146, CD90, CD106, CD29, CD71), mesenchymal stem cell-negative markers (CD19, CD45, CD31, CD18, CD56, CD34, CD14, CD80, CD40, CD86) were determined, and beta-Actin was used as an internal standard gene.
(4) Measurement Data Analysis

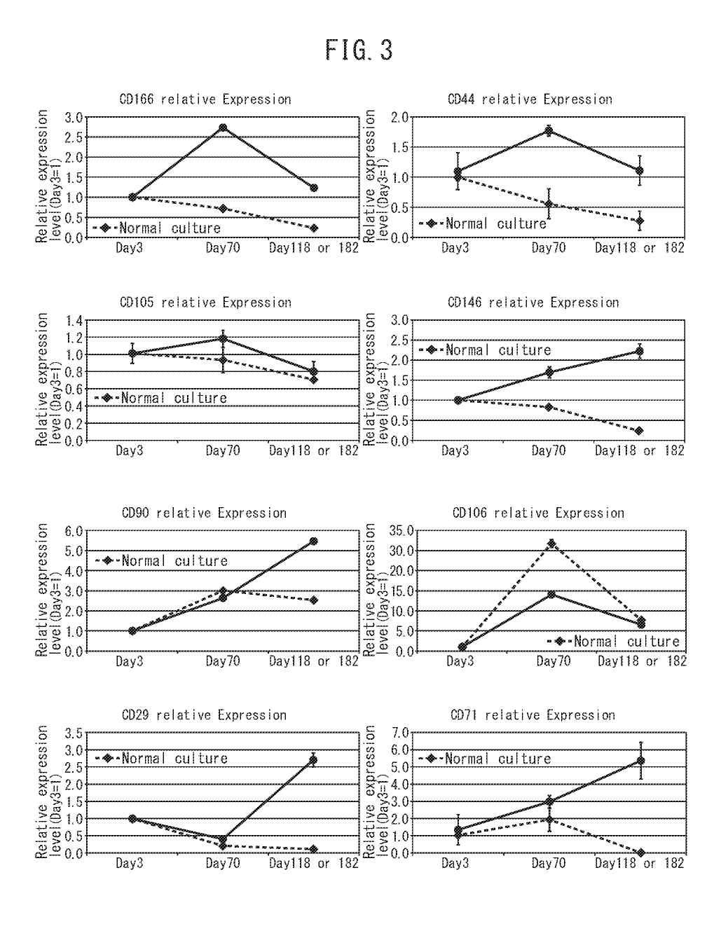

The relative expression level was calculated from the value obtained by subtracting the Ct value of each gene obtained by the measurement from the Ct value of beta-Actin measured as an internal standard gene, and comparison was made. In addition, in order to compare time-dependent changes in gene expression between the normal culture cell sample and the member culture cell sample, changes when the expression level of the cell sample cultured for 3 days was assumed to be 1 were calculated and compared. FIG. 3 depicts the results.
(5) Results

Regarding the mesenchymal stem cell-positive marker genes, the expression levels in member culture tended to be higher than those in normal culture. Regarding 5 out of 8 positive markers used for measurement, the expression levels diminished in a time-dependent manner in normal culture. This indicates that the properties of mesenchymal stem cells were lost as days elapsed during culture. Meanwhile, in the case of member culture with the use of a porous polyimide film, as the expression levels of all markers were maintained during long-term culture for 182 days, the properties of mesenchymal stem cells were retained. On the other hand, regarding the mesenchymal stem cell-negative marker genes, the expression levels in normal culture tended to be higher than those in member culture. The above indicated that differentiation of mesenchymal stem cells is suppressed by member culture.

### Example 2

Differentiation induction of human mesenchymal stem cells cultured on porous polyimide film

To a sterilized 2 cm×2 cm square vessel (Thermo Fisher Scientific, Inc., cat. 103), 0.5 ml of a stem cell culture medium (manufactured by LONZA Japan, PT-3001) was added, and a sterilized 1.4 cm size square porous polyimide film was immersed therein such that A-surface of the mesh structure of the film was faced up. Human mesenchymal stem cells were seeded at a density of 4×10⁴ cells per porous polyimide film, and the cells were continuously cultured in a CO₂ incubator while conducting medium exchange twice weekly. After the start of culture, the medium was switched to a differentiation induction medium (manufactured by PromoCell) on days 6, 70, 118, and 182.

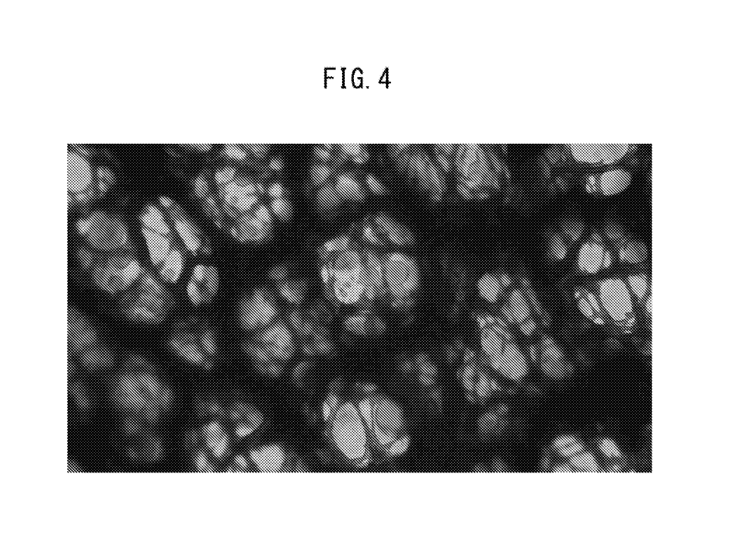

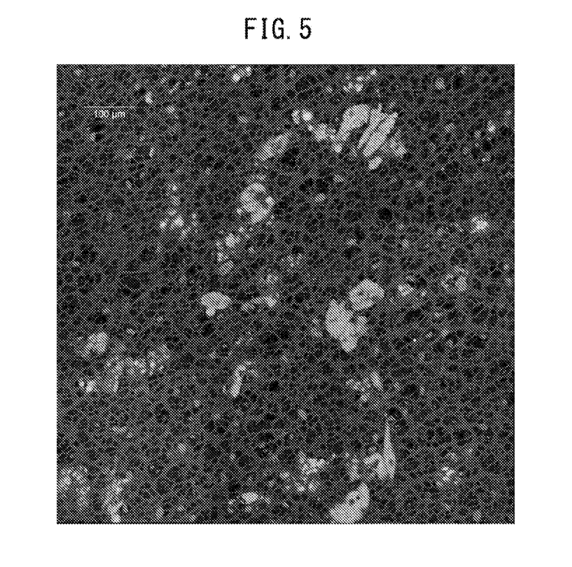

Fat globules, which were not found at the time of switching to the differentiation induction medium, began to appear around day 10 after the start of differentiation induction, and many fat globules were observed on day 14. The observed fat globules were stained with Oil red O and BODIPY to confirm induction of differentiation into adipocytes. FIG. 4 depicts a fluorescence microscopic image of staining with Oil red O of cells which were cultured for 6 days and then induced to differentiate for 13 days. FIG. 5 depicts a fluorescence microscopic image of staining with BODIPY of cells which were cultured for 182 days and then induced to differentiate for 15 days.

### Example 3

Culture of human mesenchymal stem cells on porous polyimide film To a sterilized 2 cm×2 cm square vessel (Thermo Fisher Scientific, Inc., cat. 103), 0.5 ml of a cell culture medium was added, and a sterilized 1.4 cm size square porous polyimide film was immersed therein such that A-surface of the mesh structure of the film was faced up. Mesenchymal stem cells were seeded at a density of 4×10⁴ cells per porous polyimide film, and the cells were continuously cultured in a CO₂ incubator while exchanging a medium (manufactured by GIBCO; DMEM+FBS 10%) twice weekly. The above-mentioned culture is hereinbelow referred to as “member culture,” and the obtained sample is hereinbelow referred to as “member culture cell sample.” After the start of member culture, the cell count was determined using CCK8 on days 7, 14, 21, 28, 35, and 42, and cell growth behaviors were observed.

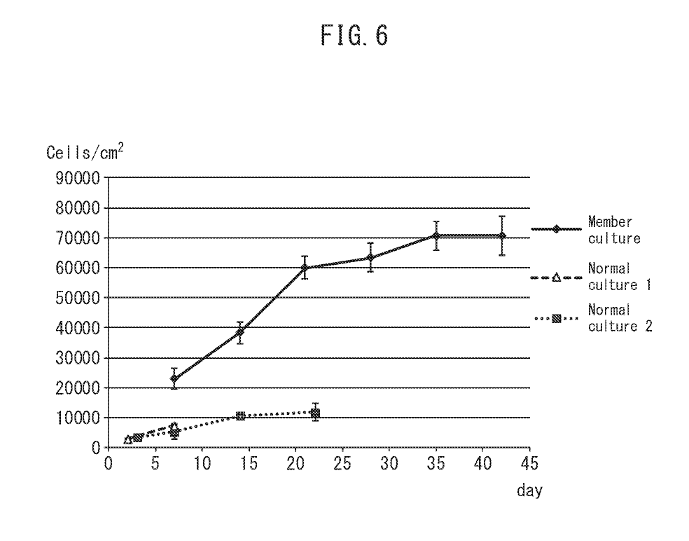

As a comparative subject, mesenchymal stem cells of the same lot as those used in member culture were cultured in a collagen type I coated dish (inner diameter: 10 cm²), the cell count was determined using CCK8, and cell growth behaviors were observed. The above-mentioned culture without the use of a porous polyimide film is hereinbelow referred to as “normal culture,” and the obtained cell sample is hereinbelow referred to as “normal culture cell sample.” Media used for member culture and normal culture and the initial number of seeded cells were listed in the following table. In addition, FIG. 6 depicts time-dependent changes in the cell counts upon member culture and normal culture of human mesenchymal stem cells. In the case of member culture, stable growth and proliferation of human mesenchymal stem cells were observed.

TABLE 2

| | | | Initial number |
|---|---|---|---|
| | | | of seeded cells |
| | Substrate | Medium | (cells/cm²) |
| Member culture | Porous | DMEM + FBS 10% | 2.0 × 10⁴ |
| | polyimide film | | |
| Normal culture 1 | Collagen type I | MSCBM | 5.0 × 10³ |
| | coated dish | | |
| Normal culture 2 | Collagen type I | DMEM + FBS 10% | 5.0 × 10³ |
| | coated dish | | |

### Example 4

Culture of Human Mesenchymal Stem Cells on Porous Polyimide Film

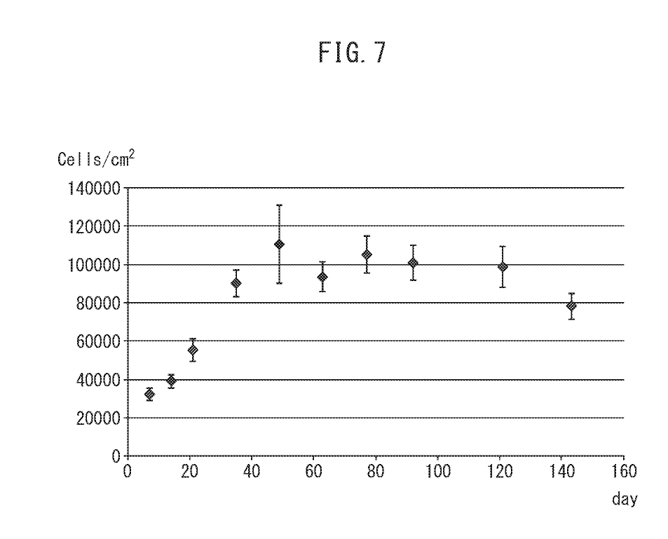

To a sterilized 2 cm×2 cm square vessel (Thermo Fisher Scientific, Inc., cat. 103), 0.5 ml of a cell culture medium (manufactured by GIBCO; DMEM+FBS 10%) was added, and a sterilized 1.4 cm size square porous polyimide film was immersed therein such that A-surface of the mesh structure of the film was faced up. Mesenchymal stem cells were seeded at a density of 4×10⁴ cells per porous polyimide film, and the cells were continuously cultured in a CO₂ incubator while conducting medium exchange twice weekly. After the start of culture, the cell count was determined using CCK8 on days 7, 14, 21, 35, 49, 63, 77, 92, 121, and 143, and cell growth behaviors were observed. FIG. 7 shows the results. In the case of member culture, stable growth and proliferation of human mesenchymal stem cells were observed for a long period of time.

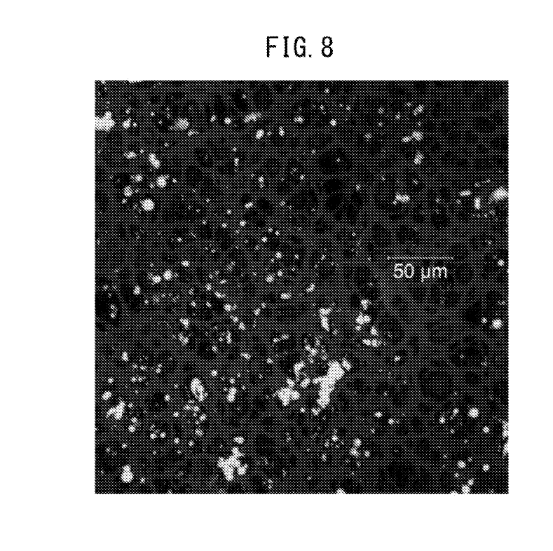

On day 125 after the start of culture, a porous polyimide film carrying engrafted cells was transferred to a vessel to which a mesenchymal stem cell/adipocyte differentiation medium 2 (manufactured by PromoCell, C-28016) was added, and culture was further continued for 10 days. Meanwhile, the induction medium was exchanged twice weekly. Thereafter, the porous polyimide film was formalin-fixed, and intracellular oil droplets formed as a result of induction by fat cells were stained with BODIPY. Even though the induction period was short, induction into adipocytes was efficiently induced, and oil droplets stained with fluorescent green color were found in the entire polyimide porous film in a distributed manner. The microscopic image is depicted in FIG. 8. The fact that differentiation-inducing ability of mesenchymal stem cells was maintained even after long-term culture demonstrated that differentiation of stem cells can be suppressed by the method of the present invention. No oil droplets stained with BODIPY were observed in member culture cell samples which were not cultured in the induction medium.

### Example 5

Culture of Human Mesenchymal Stem Cells on Porous Polyimide Film and Microscopic Observation

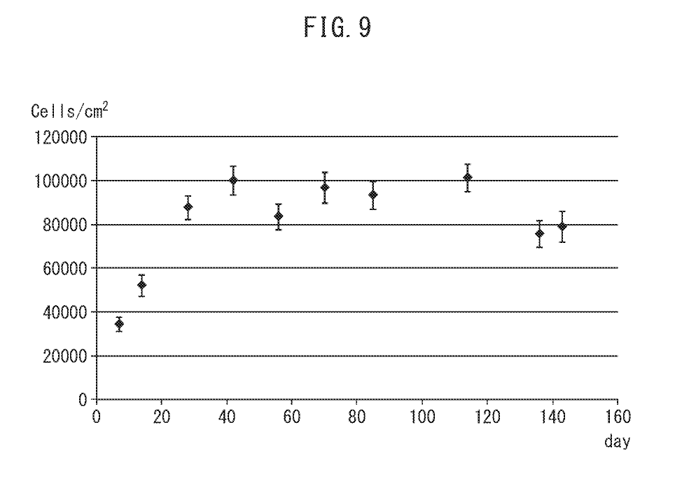

To a sterilized 2 cm×2 cm square vessel (Thermo Fisher Scientific, Inc., cat. 103), 0.5 ml of a cell culture medium (manufactured by GIBCO; DMEM+FBS 10%) was added, and a sterilized 1.4 cm size square porous polyimide film was immersed therein such that A-surface of the mesh structure of the film was faced up. Mesenchymal stem cells were seeded at a density of 4×10⁴ cells per porous polyimide film, and the cells were continuously cultured in a CO₂ incubator while conducting medium exchange twice weekly. After the start of culture, the cell count was determined using CCK8 on days 7, 14, 28, 42, 56, 70, 85, 114, 136, and 143, and cell growth behaviors were observed. FIG. 9 shows the results. In the case of member culture, stable growth and proliferation of human mesenchymal stem cells were observed for a long period of time.

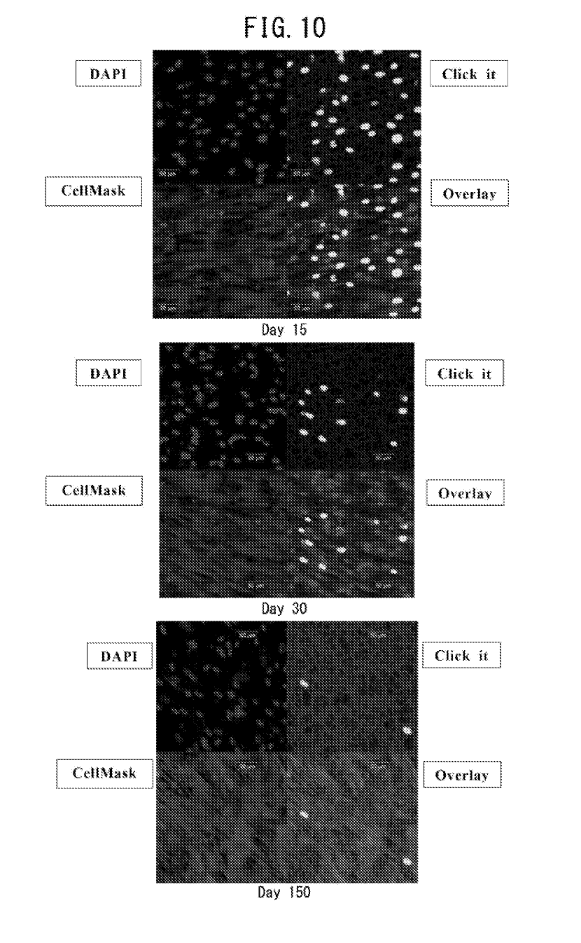

On days 15, 30, and 150 after the start of culture, the porous polyimide film carrying engrafted cells was fixed and stained with a Click-iT (trademark) EdU Alexa Fluor (trademark) 488 imaging reagent (manufactured by Thermo Fisher Scientific, Inc.), CellMask Orange Plasma Membrane Stain, and DAPI, and fluorescence microscope images were taken to evaluate DNA synthesizing ability of the cells. The fluorescence microscopic image is depicted in FIG. 10. It was revealed that there was a certain proportion of Edu-positive cells capable of synthesizing DNA synthesis even after long-term culture.

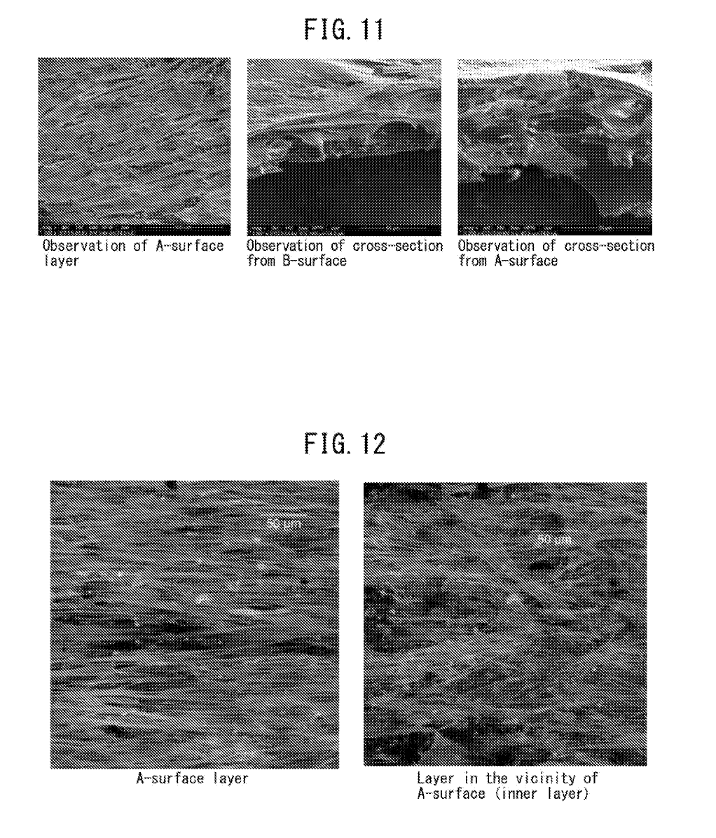

On day 146 after the start of culture, the porous polyimide film carrying engrafted cells was formalin-fixed and electron microscopic observation was performed. Specifically, after fixing the porous polyimide film with a mixed fixative solution of 2.5% glutaraldehyde and 2% formaldehyde, post-fixation with osmium tetroxide was carried out, dehydration was conducted by a sequential ethanol substitution method, and then, freeze fracturing was performed at the liquid nitrogen temperature. After vacuum freeze-drying using t-butyl alcohol, antistatic treatment was carried out by osmium plasma vapor deposition, and observation by scanning electron microscopy (SEM) was conducted. For observation, a field emission type SEM was used, and a secondary electron image obtained under an acceleration voltage of 5 kV and high vacuum was observed. FIG. 11 shows the results. Many interesting member culture behaviors such as cell alignment and formation of a layered structure of cells were observed.

On day 150 after the start of culture, a porous polyimide film carrying engrafted cells was formalin-fixed, and fluorescence microscopic observation was conducted. Specifically, after the porous polyimide film was formalin-fixed, staining with Alexa Fluor (registered trademark) 488 phalloidin, CellMask Orange Plasma Membrane Stain, and DAPI was performed, and a fluorescence microscopic image was obtained by a confocal laser microscope. FIG. 12 shows the results. Two different regions of the A-surface layer and the layer in the vicinity of A-surface (inner layer) were measured independently, and the state of cell aggregation was verified. In the A-surface layer, as with the results of SEM observation, strong orientation of the cells in one direction was observed, whereas in the layer in the vicinity of A-surface (inner layer), it was observed that the orientation disappeared and cells strongly adhered to the mesh surface of the film.

### Example 6

Long-Term Culture of Human Mesenchymal Stem Cells on Porous Polyimide Film and Differentiation Induction of Culture Cells

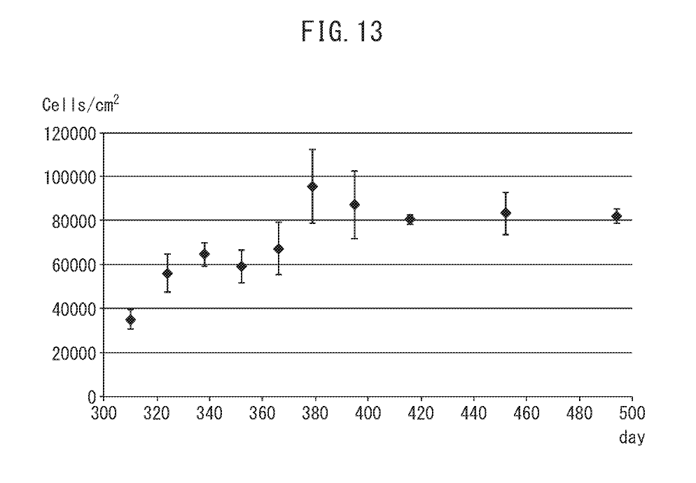

To a sterilized 2 cm×2 cm square vessel (Thermo Fisher Scientific, Inc., cat. 103), 0.5 ml of a stem cell culture medium (manufactured by LONZA; mesenchymal stem cell medium MSCBM) was added, and a sterilized 1.4 cm size square porous polyimide film was immersed therein such that A-surface of the mesh structure of the film was faced up. Mesenchymal stem cells were seeded at a density of 4×10⁴ cells per porous polyimide film, and the cells where cultured in a CO₂ incubator for 14 days while conducting medium exchange twice weekly. Thereafter, 10 sheets of the porous polyimide film carrying engrafted cells were each transferred to a 20 cm² dish to which 4 ml of an MSCBM medium was added, followed by culture. After culture for 310 days, each porous polyimide film was transferred to a sterilized 2 cm×2 cm square vessel containing a cell culture medium, and culture was continued. The cell count was determined using CCK8, and cell growth behaviors were observed. FIG. 13 shows the results. After the transfer to the square vessel, stable growth and proliferation of human mesenchymal stem cells were observed.

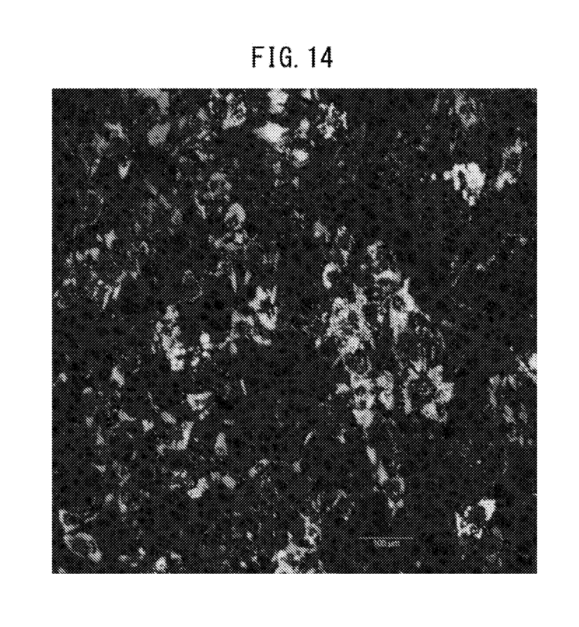

On day 463 after the start of culture, each porous polyimide film carrying engrafted cells was transferred to a vessel to which a mesenchymal stem cell/adipocyte differentiation medium 2 (manufactured by PromoCell, C-28016) was added, and culture was further continued for 26 days. Meanwhile, the induction medium was exchanged twice weekly. Thereafter, the porous polyimide film was formalin-fixed, and intracellular oil droplets formed as a result of induction by fat cells were stained with BODIPY. The microscopic image by a confocal laser microscope is depicted in FIG. 14. Induction into adipocytes was efficiently induced, and oil droplets stained with fluorescent green color were found in the entire polyimide porous film in a distributed manner.

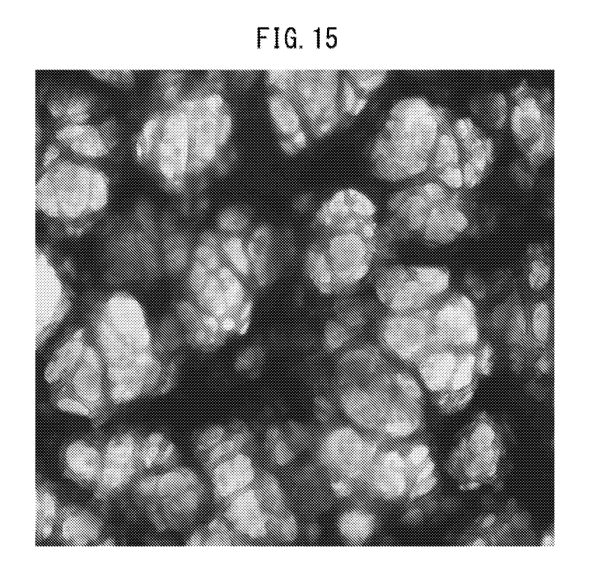

In addition, on day 490 after the start of culture, each porous polyimide film carrying engrafted cells was transferred to a vessel to which an osteoblast differentiation induction medium (manufactured by PromoCell, C-28013) was added, and culture was further continued for 26 days. Thereafter, the porous polyimide film was transferred to a vessel to which an osteoblast mineralization medium (manufactured by PromoCell, C-28020) was added, and mineralization induction was performed for 14 days. Thereafter, staining was carried out with a mineralization staining kit manufactured by Cosmo Bio, and remarkable reddening of the mineralized part was observed with a microscope. The optical microscopic image is depicted in FIG. 15.

The results of induction from mesenchymal stem cells to adipocytes or osteoblasts confirmed that mesenchymal stem cells maintain differentiation-inducing ability even after long-term culture.

### Example 7

Gene Analysis of Mesenchymal Stem Cells Cultured on Porous Polyimide Film for Long Period of Time

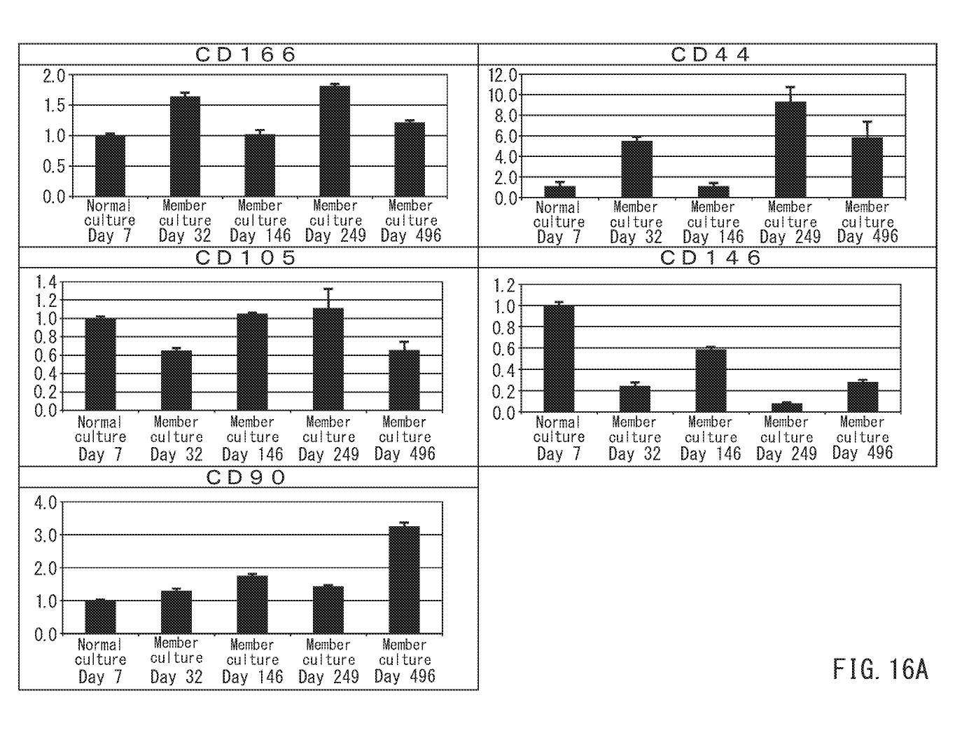

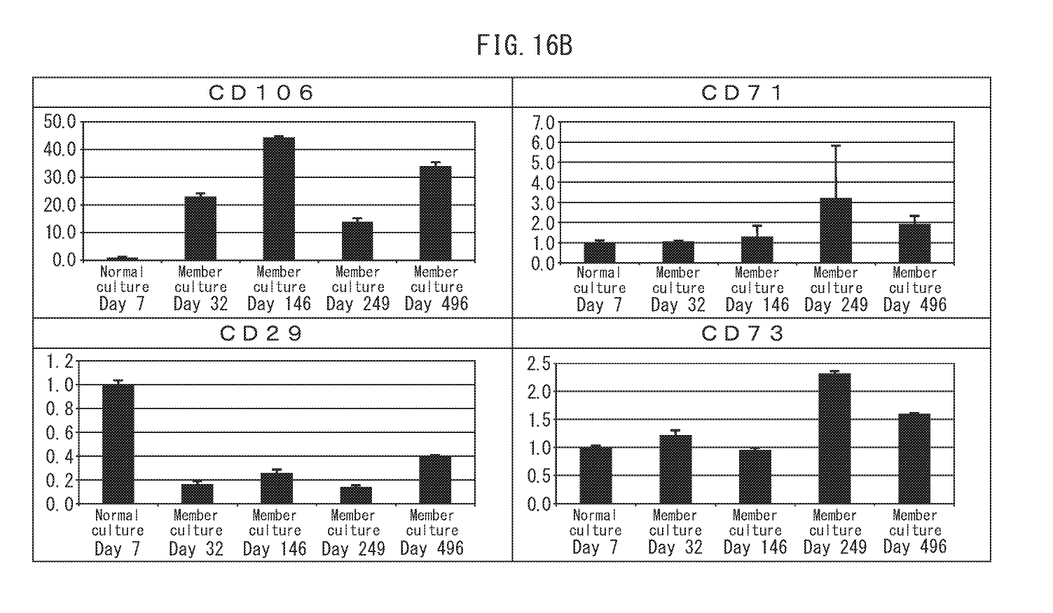

To a sterilized 2 cm×2 cm square vessel (Thermo Fisher Scientific, Inc., cat. 103), 0.5 ml of a stem cell culture medium (manufactured by LONZA; mesenchymal stem cell medium MSCBM) was added, and a sterilized 1.4 cm size square porous polyimide film was immersed therein such that A-surface of the mesh structure of the film was faced up. Mesenchymal stem cells were seeded at a density of 4×10⁴ cells per porous polyimide film, and the cells where cultured in a CO₂ incubator for 14 days while conducting medium exchange twice weekly. Thereafter, 10 sheets of the porous polyimide film carrying engrafted cells were each transferred to a 20 cm² dish to which 4 ml of an MSCBM medium was added, followed by culture. Gene analysis of the member culture cell sample on day 249 after the start of culture was conducted in the same manner as in Example 1. In addition, gene analysis was also conducted in the same manner as in Example 1 for the member culture cell sample on day 32 after the start of culture in Example 4, the member culture cell sample on day 146 after the start of culture in Example 5, and the member culture cell sample on day 496 after the start of culture in Example 6. FIGS. 16A and 16B depict the results concerning positive markers. For comparison, the expression level of each gene in the cell sample treated by normal culture for 7 days without the use of the porous polyimide film was assumed to be 1. The expression levels of all negative markers were confirmed to be low. It was confirmed that the properties of mesenchymal stem cells can be maintained using the method of the present invention even after long-term culture.

### Example 8

Gene Analysis of Human Type II Alveolar Epithelial Cells Cultured on Porous Polyimide Film
1. Sample Preparation

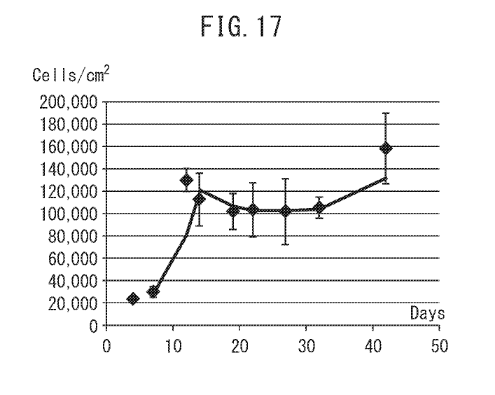

A sterilized 1.4 cm size square porous polyimide film was allowed to stand still in a sterilized 2 cm×2 cm square vessel (Thermo Fisher Scientific, Inc., cat. 103) such that A-surface of the mesh structure of the film was faced up. A cryopreservation solution of 20-week-old fetus-derived type II alveolar epithelial cells was thawed and the type II alveolar epithelial cells were suspended in an alveolar epithelial cell medium (manufactured by ScienCell Research Laboratories; product code: 3201), and the cells in the form of suspension cell droplets were placed on a dry porous polyimide film at a density of 1×10⁵ cells per porous polyimide film, followed by spontaneous passage of the liquid part (suction seeding method). After the passage of the liquid part, 1 ml of a medium was additionally injected, and culture was continuously performed in a CO₂ incubator. The medium (1 ml) was exchanged twice weekly. After the start of culture, the cell count was periodically evaluated using CCK8, and the cell growth condition was confirmed. FIG. 17 depicts changes in the cell count. Cells cultured for 4 days and cells cultured for 32 days were each used as a gene analysis sample. The above-mentioned culture with the use of a porous polyimide film is hereinbelow referred to as “member culture,” and the obtained cell sample is hereinbelow referred to as “member culture cell sample.”

In addition, type II alveolar epithelial cells were cultured under the same conditions except that no porous polyimide film was used. Cells cultured for 4 days were used as a gene analysis sample. The above-mentioned culture without the use of a porous polyimide film is hereinbelow referred to as “normal culture,” and the obtained sample is hereinbelow referred to as “normal culture cell sample.”
2. Gene Analysis

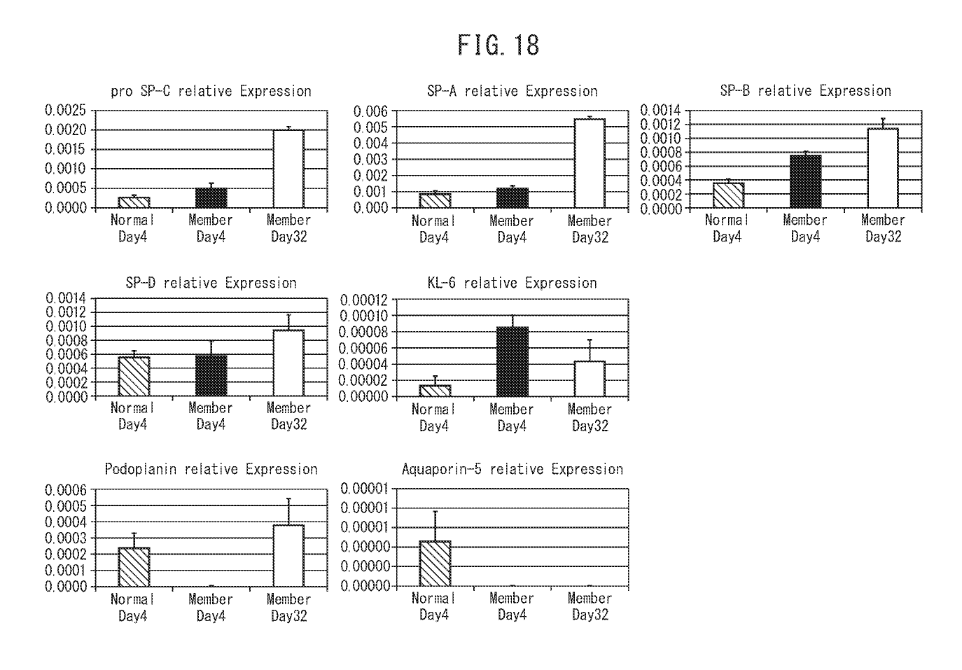

Gene analysis was conducted for the obtained samples by the same procedures as in Example 1. The expression levels of type II alveolar epithelial cell-positive markers (Pro SP-C, SP-A, SP-B, SP-D, KL-6) and type I alveolar epithelial cell-positive markers (Podoplanin), Aquaporin-5 were determined. FIG. 18 shows the results.
3. Results

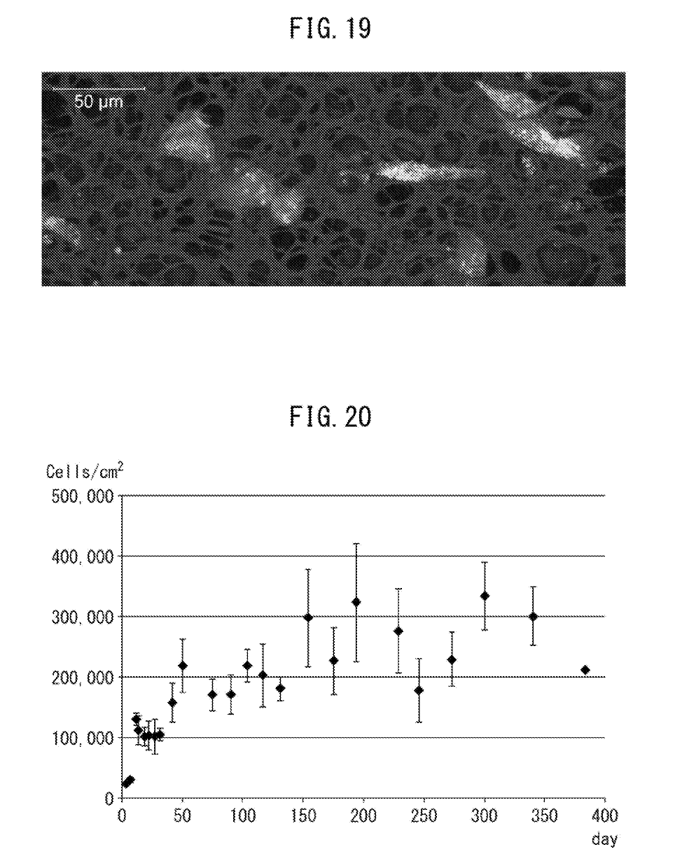

Regarding the type II alveolar epithelial cell-positive marker genes, the expression levels in member culture tended to be higher than those in normal culture. This suggests that differentiation of type II alveolar epithelial cells into type I alveolar epithelial cells was suppressed by member culture. In addition, it was suggested that type II alveolar epithelial cells increase as the culture cell count increases, and the increased level was is maintained. In order to verify the above, Pro SP-C and Podoplanin were evaluated by immunostaining (Day 22). As a result, similar results were confirmed (FIG. 19).

### Example 9

Long-term culture of human type II alveolar epithelial cells on porous polyimide film Cell culture in Example 8 was continued for about 1 year. FIG. 20 depicts the results. Stable growth and proliferation of type II alveolar epithelial cells were observed for a long period of time.

### Example 10

Gene analysis of human type II alveolar epithelial cells cultured on porous polyimide film for long period of time
1. Sample Preparation

A sterilized 1.4 cm size square porous polyimide film was allowed to stand still in a sterilized 2 cm×2 cm square vessel (Thermo Fisher Scientific, Inc., cat. 103) such that A-surface of the mesh structure of the film was faced up. A cryopreservation solution of 20-week-old fetus-derived type II alveolar epithelial cells was thawed, thereby preparing a medium suspension liquid. The cells were seeded on a porous polyimide film by the following three types of methods.

Seeding method 1: The cells in the form of a suspension liquid were added on a porous polyimide film which was moistened with a medium in advance at a density of 2×10⁴ cells per porous polyimide film (natural seeding method).

Seeding method 2: The cells in the form of a suspension liquid were added on a porous polyimide film which was moistened with a medium in advance at a density of 4×10⁴ cells per porous polyimide film (natural seeding method).

Seeding method 3: the cells in the form of suspension cell droplets were placed on a dry porous polyimide film at a density of 1×10⁵ cells per porous polyimide film, followed by spontaneous passage of the liquid part (suction seeding method).

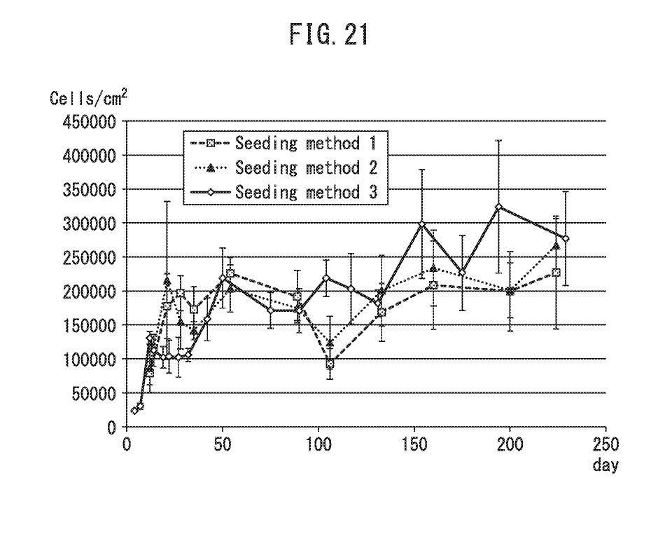

After cell seeding by the above-mentioned, the cells were continuously cultured in a CO₂ incubator while conducting medium exchange twice weekly. The cell count was periodically evaluated using CCK8, and the cell growth condition was confirmed. FIG. 21 shows changes in the cell count.
2. Gene Analysis

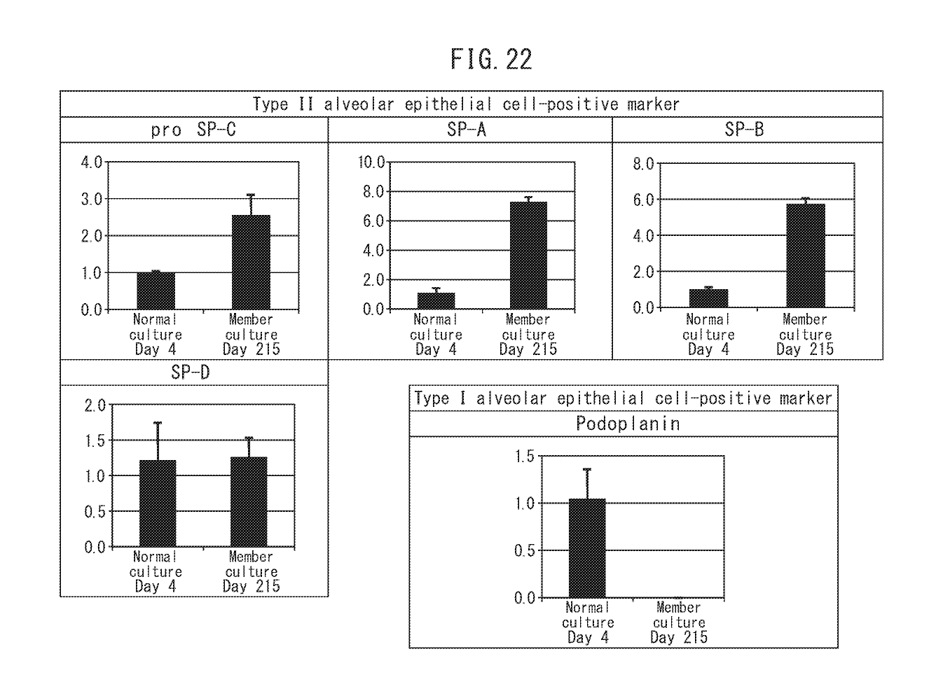

Cell seeding was carried out by the above-mentioned seeding method 3, and gene analysis of the member culture cell sample cultured for 215 days was conducted. As a control sample, the normal culture cell sample in Example 9 was used. FIG. 22 depicts the results. Regarding the type II alveolar epithelial cell-positive marker genes, the expression levels in member culture tended to be higher than those in normal culture. This suggests that differentiation of type II alveolar epithelial cells into type I alveolar epithelial cells was suppressed by member culture.

### INDUSTRIAL APPLICABILITY

The present invention can be utilized to suppress differentiation of stem cells so as to supply the stem cells in large amounts.

1. A method for suppressing the differentiation of stem cells, the method comprising the steps of:
(1) applying the stem cells to a porous polymer film; and
(2) culturing and proliferating the stem cells;
wherein the porous polymer film is a three-layer structure porous polymer film having a surface layer A and a surface layer B, the surface layers having a plurality of pores, and a macrovoid layer sandwiched between the surface layers A and B;
wherein an average pore diameter of the pores present in the surface layer A is smaller than an average pore diameter of the pores present in the surface layer B;
wherein the macrovoid layer has a partition wall bonded to the surface layers A and B, and a plurality of macrovoids surrounded by the partition wall and the surface layers A and B; and
wherein the pores in the surface layers A and B communicate with the macrovoids.

2. The method according to claim 1, wherein the cells are ES cells, EC cells, EG cells, nuclear transfer ES cells, ntES cells, iPS cells, hematopoietic stem cells, mesenchymal stem cells, hepatic stem cells, pancreatic stem cells, skin stem cells, bone marrow stem cells, muscle stem cells, germ stem cells, spermatogonia, type II alveolar epithelial cells, adipose stem cells, dental pulp stem cells, dedifferentiated adipocytes or MUSE cells.

3. The method according to claim 1 or 2, wherein the step (2) is carried out for at least 30 days.

4. The method according to any one of claims 1 to 3, wherein in the step (2), the cells are allowed to proliferate to 1.0×10⁵ or higher per square centimeter of the porous polymer film.

5. The method according to any one of claims 1 to 4, wherein two or more porous polymer films are layered either above and below or left and right in the cell culture medium.

6. The method according to any one of claims 1 to 5, wherein the porous polyimide film is
i) folded,
ii) wound into a roll-like shape,
iii) connected as sheets or pieces with a filamentous structure, or
iv) bound into a rope-like shape, and suspended or fixed in a cell culture medium in a cell culture vessel.

7. The method according to any one of claims 1 to 6, wherein, in step (2), a part or all of a porous polyimide film is not in contact with a liquid phase of a cell culture medium.

8. The method according to any one of claims 1 to 7, wherein in step (2), the total volume of the cell culture medium contained in the cell culture vessel is 10000 times or lower than the sum of the porous polyimide film volume comprising a cell viable region.

9. The method according to any one of claims 1 to 8, wherein an average pore diameter of the surface layer A is 0.01 to 50 μm.

10. The method according to any one of claims 1 to 9, wherein an average pore diameter of the surface layer B is 20 to 100 μm.

11. The method according to any one of claims 1 to 10, wherein a film thickness of the porous polymer film is 5 to 500 μm.

12. The method according to any one of claims 1 to 11, wherein the porous polymer film is a porous polyimide film.

13. The method according to claim 12, wherein the porous polyimide film is a porous polyimide film comprising a polyimide derived from tetracarboxylic dianhydride and diamine.

14. The method according to claim 12 or 13, wherein the porous polyimide film is a colored porous polyimide film that is obtained by molding a polyamic acid solution composition comprising a polyamic acid solution derived from tetracarboxylic dianhydride and diamine, and a coloring precursor, and subsequently heat-treating the resultant composition at 250° C. or higher.

15. The method according to any one of claims 1 to 11, wherein the porous polymer film is a porous polyethersulfone film.

16. A method for preparing stem cells, the method comprising the steps of:
(1) applying the stem cells to a porous polymer film; and
(2) culturing and proliferating the stem cells;
wherein the porous polymer film is a three-layer structure porous polymer film having a surface layer A and a surface layer B, the surface layers having a plurality of pores, and a macrovoid layer sandwiched between the surface layers A and B;
wherein an average pore diameter of the pores present in the surface layer A is smaller than an average pore diameter of the pores present in the surface layer B;
wherein the macrovoid layer has a partition wall bonded to the surface layers A and B, and a plurality of macrovoids surrounded by the partition wall and the surface layers A and B; and
wherein the pores in the surface layers A and B communicate with the macrovoids; and
wherein in the step (2), the differentiation of the cells is suppressed.

17. A method for inducing the differentiation of stem cells, the method comprising the steps of:
(1) applying the stem cells to a porous polymer film;
(2) culturing and proliferating the stem cells; and
(3) culturing the cultured stem cells under differentiation-inducing conditions;
wherein the porous polymer film is a three-layer structure porous polymer film having a surface layer A and a surface layer B, the surface layers having a plurality of pores, and a macrovoid layer sandwiched between the surface layers A and B;
wherein an average pore diameter of the pores present in the surface layer A is smaller than an average pore diameter of the pores present in the surface layer B;
the macrovoid layer has a partition wall bonded to the surface layers A and B, and a plurality of macrovoids surrounded by the partition wall and the surface layers A and B;
wherein the pores in the surface layers A and B communicate with the macrovoids; and
wherein in the step (2), the differentiation of the stem cells is suppressed.
